(19) United States
(12) Patent Application Publication
(10) Pub. No.: US 2006/0094031 A1
(43) Pub. Date: May 4, 2006

(54) Alpha-L-arabinofuranosidase histochemical reporter gene assay

(75) Inventor: Brian L. Sauer, Kansas City, MO (US)

Correspondence Address: POLSINELLI SHALTON WELTE SUELTHAUS P.C., 700 W. 47TH STREET, SUITE 1000, KANSAS CITY, MO, 64112-1802, US

(73) Assignee: Stowers Institute for Medical Research

(21) Appl. No.: 11/066,633

(22) Filed: Feb. 25, 2005

Related U.S. Application Data
(60) Provisional application No. 60/625,432, filed on Nov. 4, 2004.

(51) Int. Cl. C12Q 1/68; C07H 21/04; C12N 1/21; C12N 5/06; C12N 1/18

(52) U.S. Cl. 435/6; 435/252.3; 435/254.2; 435/325; 536/24.3; 536/25.32

(57) ABSTRACT

The present invention provides a novel reporter gene assay. In particular, the present invention provides a histochemical reporter gene assay employing α-_{L}-arabinofuranosidase in combination with a chromogenic α-_{L}-arabinofuranosidase substrate. The α-_{L}-arabinofuranosidase histochemical reporter assay may be utilized to characterize gene regulatory regions of interest either alone or in combination with other assays.

### CROSS-REFERENCE TO RELATED APPLICATIONS

This application claims priority from Provisional Application Ser. No. 60/625,432 filed on Nov. 4, 2004, which is hereby incorporated by reference in its entirety.

### FIELD OF THE INVENTION

The present invention relates to a reporter gene assay. In particular, the present invention provides a histochemical reporter gene assay employing α-_{L}-arabinofuranosidase in combination with a chromogenic α-_{L}-arabinofuranosidase substrate. The α-_{L}-arabinofuranosidase may also be used as an enzyme label in immunological diagnostic and immunohistochemical reactions.

### BACKGROUND OF THE INVENTION

Recombinant DNA and gene transfer techniques have become powerful tools for monitoring gene expression and understanding gene regulation in biological assays. Central to these techniques is the use of reporter gene assays to study a gene regulatory element of interest, such as a promoter. Reporter gene assays are useful for studying gene regulatory elements because reporter gene activity (i.e., production of the reporter protein) is directly proportional to transcriptional activity of the regulatory elements of the gene. In particular, reporter gene assays provide a means to identify sequences and factors that control gene expression at the transcriptional level. For example, analysis of constructs containing various deletions within the regulatory region enables mapping of regulatory sequences necessary for transcription and cell specific expression. Other uses for reporter gene assays include: identification of sequences and factors that control genes at the translational level, study of mechanisms and factors that influence and alter gene expression levels and drug screening in cell-based assays.

A reporter gene construct for use in reporter assays typically includes a reporter gene fused to one or more gene regulatory elements that are of interest. Most often the reporter gene encodes an easily assayed enzyme that is normally absent or present at low levels, in the cell or organism being examined. In addition, the reporter protein typically has a unique enzymatic activity or structure that enables it to be distinguished from other proteins that are present.

To provide relevant experimental information, reporter assays must be sensitive, thus enabling the detection of low levels of reporter protein in cell lines that transfect poorly. The sensitivity of a reporter gene assay is a function of the detection method as well as reporter mRNA and protein turnover, and endogenous (background) levels of the reporter activity.

Commonly used reporter assay detection techniques use isotopic, calorimetric, fluorometric or luminescent enzyme substrates and immunoassay-based procedures with isotopic or color endpoints. Many of these systems, however, have disadvantages that limit their usefulness in these assays. For example, the cost, sensitivity and inconvenience of using radioisotopes limit isotopic substrates and immunoassays. Fluorometric systems require external light sources which must be filtered to discriminate fluorescent signal, thereby limiting the sensitivity and increasing complexity of the detection system. Furthermore, fluorescence from endogenous source can interfere with fluorometric measurements. Some colorimetric systems lack the sensitivity desired for sensitive reporter gene assays.

Several genes that encode reporter proteins are currently used including chloramphenicol acetyltransferase (CAT), secreted alkaline phosphatase, luciferase, β-galactosidase, β-glucuronidase, α-_{L}-arabinofuranosidase, and human growth hormone, among others. β-galactosidase and CAT are two of the most widely used reporter genes. In particular, β-galactosidase from Escherichia coli, the product of the lacZ gene, has become an ubiquitous tool for monitoring gene expression in a wide variety of both prokaryotes and eukaryotes. β-galactosidase detection is commonly performed with calorimetric substrates that, depending upon the application, can lack sensitivity. Not infrequently, for example, the level of endogenous β-galactosidase activity in mammalian cells and several bacterial strains is sufficiently high so as to obscure lacZ reporter expression. Fluorescent substrates are also used to detect β-galactosidase, however, those assays also lacks sensitivity and are limited by background autofluorescence and signal quenching. α-_{L}-arabinofuranosidase has successfully been used in combination with the chromogenic substrate 5-bromo-3-indolyl-α-_{L}-arabinofuranoside to detect reporter gene activity in bacterial colonies (4). But α-_{L}-arabinofuranosidase has not been utilized to detect reporter gene activity in mammalian cells or as a part of a dual reporter system to detect the activity of two reporter genes in the same cell. The most widely used assay for CAT is radioisotopic, but it exhibits only moderate sensitivity and suffers from a narrow dynamic range. β-Glucuronidase (GUS) is a widely used reporter gene in plant genetic research and to a lesser extent in mammalian cells. A common assay for GUS uses a fluorescent substrate, but this system is limited by background autofluorescence and signal quenching. Luciferase has become a more widely used reporter gene as it is quantitated using a very sensitive bioluminescent assay utilizing the substrate, luciferin.

Sensitive chemiluminescent assays, not limited to reporter gene assays, have been described using dioxetane substrates. These dioxetane substrates emit visible light following enzyme-induced degradation. Enhancement of the chemiluminescent degradation of 1,2-dioxetanes by enhancer substances comprised of certain water-soluble substances, such as globular proteins that have hydrophobic regions, has been described (see, e.g., Voyta et al., U.S. Pat. No. 5,145,772, incorporated herein by reference). These dioxetane substrates are also used in reporter gene assays for alkaline phosphatase, β-galactosidase, and β-glucuronidase. But no reporter gene assay using dioxetane substrates has been described in which the products of multiple reporter genes are sequentially quantitated in the same cell.

A need, therefore, exists for a reporter gene assay that is simple, rapid, highly sensitive and that has a reporter protein that can be detected without the use of either radioisotopes or external light sources. In particular, a need exists for a reporter gene assay that is sensitive when used in mammalian cells. Moreover, under certain circumstances, it would be desirable to have a reporter gene assay that can be used in conjunction with another reporter gene assay to be able to follow expression of two different reporter genes in the same cell or organism and in particular, within the same mammalian cell.

### SUMMARY OF THE INVENTION

Briefly, the present invention provides a histochemical reporter gene assay employing α-_{L}-arabinofuranosidase in combination with a chromogenic α-_{L}-arabinofuranosidase substrate. Reporter gene activity can readily be detected because when the chromogenic α-_{L}-arabinofuranosidase substrate is contacted with α-_{L}-arabinofuranosidase, a colored product is formed. The reporter gene assay may be used alone for the histochemical detection of gene expression in a cell or organism. Alternatively, the reporter gene assay may be used in combination with another reporter assay, such as with β-galactosidase, to permit histochemical detection of the expression of two genes in a cell or organism. Advantageously, unlike β-galactosidase and other reporter genes, neither the α-_{L}-arabinofuranosidase enzyme nor its substrates are naturally present in mammalian cells, thus making the reporter gene assay of the present invention particularly suitable for use in mammalian cells.

Among the several aspects of the invention, therefore, is the provision of a histochemical reporter gene assay comprising a vector having a nucleic acid encoding an α-_{L}-arabinofuranosidase enzyme and a chromogenic α-_{L}-arabinofuranosidase substrate. In certain embodiments, the α-_{L}-arabinofuranosidase nucleic acid is from Bacillus subtilis or Aspergillus niger and the substrate is a chromogenic α-_{L}-arabinofuranosidase substrate. In other embodiments, the α-_{L}-arabinofuranosidase nucleic acid is from Steptomyces livians and the chromogenic substrate is a compound having formula (I): 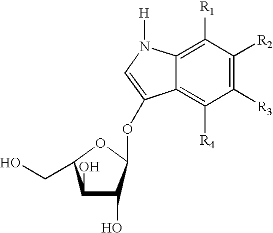
wherein:

R₁, R₂ and R₄ are independently selected from the group consisting of bromo, chloro, and hydrogen; and

R₃ is chloro or hydrogen.

Yet another aspect of the invention encompasses a cell comprising a vector having a nucleic acid encoding an α-_{L}-arabinofuranosidase enzyme and a chromogenic α-_{L}-arabinofuranosidase substrate. In certain embodiments, the α-_{L}-arabinofuranosidase nucleic acid is from Bacillus subtilis or Aspergillus niger and the substrate is a chromogenic α-_{L}-arabinofuranosidase substrate. In other embodiments, the α-_{L}-arabinofuranosidase nucleic acid is from Steptomyces livians and the chromogenic substrate is a compound having formula (I): 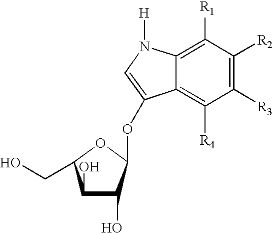
wherein:

R₁, R₂ and R₄ are independently selected from the group consisting of bromo, chloro, and hydrogen; and

R₃ is chloro or hydrogen.

An additional aspect of the invention provides a transgenic non human organism having a cell comprising a vector having a nucleic acid encoding an α-_{L}-arabinofuranosidase enzyme and a chromogenic α-_{L}-arabinofuranosidase substrate. In certain embodiments, the α-_{L}-arabinofuranosidase nucleic acid is from Bacillus subtilis or Aspergillus niger and the substrate is a chromogenic α-_{L}-arabinofuranosidase substrate. In other embodiments, the α-_{L}-arabinofuranosidase nucleic acid is from Steptomyces livians and the chromogenic substrate is a compound having formula (I): 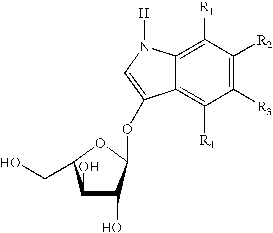
wherein:

R₁, R₂ and R₄ are independently selected from the group consisting of bromo, chloro, and hydrogen; and

R₃ is chloro or hydrogen.

Yet another aspect of the invention encompasses a chromogenic α-_{L}-arabinofuranosidase substrate having an indolyl substituted with a halogen and an α-_{L}-arabinofuranoside, wherein the chromogenic substrate is other than 5-bromo-3-indolyl-α-_{L}-arabinofuranoside. When the chromogenic substrate is contacted with α-_{L}-arabinofuranosidase, a colored product is formed. In certain embodiments, the chromogenic α-_{L}-arabinofuranosidase substrate is selected from the group of compounds consisting of 5-bromo-4-chloro-3-indolyl-α-_{L}-arabinofuranoside; 6-chloro-3-indolyl-α-_{L}-arabinofuranoside; 4,6-dichloro-3-indolyl-α-_{L}-arabinofuranoside; 6,7-dichloro-3-indolyl-α-_{L}-arabinofuranoside; and 4,6,7-trichloro-3-indolyl-α-_{L}-arabinofuranoside. In other embodiments, the chromogenic α-_{L}-arabinofuranosidase substrate is a compound having formula (I): 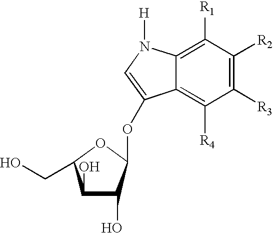
wherein:

R₁, R₂ and R₄ are independently selected from the group consisting of bromo, chloro, and hydrogen; and

R₃ is chloro or hydrogen.

A further aspect of the invention provides a composition for use in monitoring transcriptional activity of a regulatory sequence. In a typical embodiment, the composition comprises a chromogenic α-_{L}-arabinofuranosidase substrate having an indolyl substituted with a halogen and an α-_{L}-arabinofuranoside, wherein the compound is other than 5-bromo-3-indolyl-α-_{L}-arabinofuranoside; and a chromogenic β-galactosidase substrate.

An additional aspect of the invention encompasses a kit for monitoring the transcriptional activity of a regulatory sequence. The kit generally comprises a vector having an insertion site for a regulatory element, wherein the regulatory element is operably linked to a nucleic acid encoding α-_{L}-arabinofuranosidase; a chromogenic α-_{L}-arabinofuranosidase substrate; and instructions for detecting the activity of α-_{L}-arabinofuranosidase.

Yet another aspect of the invention is directed to a method for monitoring the transcriptional activity of a regulatory sequence in a cell. In general, the method comprises introducing into the cell a vector comprising the regulatory sequence operably linked to a nucleic acid encoding α-_{L}-arabinofuranosidase and detecting the activity of α-_{L}-arabinofuranosidase in the cell, wherein the activity of α-_{L}-arabinofuranosidase is directly proportional to the transcriptional activity of the regulatory element. In certain embodiments, the α-_{L}-arabinofuranosidase is from Bacillus subtilis or Aspergillus niger. In other embodiments, the α-_{L}-arabinofuranosidase nucleic acid is from Steptomyces livians.

Other aspects and features of the invention are described in more detail below.

### BRIEF DESCRIPTION OF THE FIGURES

FIG. 1 depicts a schematic of the CMV abfA expression vector. Several unique restriction sites are shown that may be utilized to facilitate subcloning of the abfA gene.

FIG. 2 depicts a series of images showing chromogenic detection of β-galactosidase and α-_{L}-arabinofuranosidase activities in NIH 3T3 mammalian cells.
2(A) depicts a chromogenic image showing cells transfected with CMV-abfA and visualized with Z-ara. Shown is a representative field viewed using differential interference contrast (DIC) at 400×.
2(B) depicts a birefringence image of abfA positive cells. The same field in (A) was viewed using polarized light.
2(C) and (D) depicts a chromogenic image illustrating dual detection of lacZ and abfA. Cells were cotransfected with CMV-abfA and proBGN (7), an expression plasmid encoding a nuclear-localizing β-galactosidase. Processing was as in (A), but with the addition of magenta-gal to the in situ enzyme activity detection step. Cells were viewed at 200× either by DIC (C) or without DIC (D).

FIG. 3 depicts a series of images showing chromogenic detection of β-galactosidase and α-_{L}-arabinofuranosidase activities in CHO K1 mammalian cells. Cells were cotransfected with CMV-abfA and proBGN (7), an expression plasmid encoding a nuclear-localizing β-galactosidase. CMV-abfA was visualized with Z-ara and lacZ was visualized with X-gal. Shown is a representative field viewed using differential interference contrast (DIC) at 400×.

### DETAILED DESCRIPTION OF THE PREFERRED EMBODIMENTS

The present discovery provides a histochemical reporter gene assay employing α-_{L}-arabinofuranosidase in combination with a chromogenic α-_{L}-arabinofuranosidase substrate. The reporter gene assay may be used to characterize the transcriptional activity of a regulatory sequence operably linked to the reporter gene. Because reporter gene activity (i.e., production of the reporter protein) is directly proportional to transcriptional activity of the regulatory sequences, the abfA reporter gene can be utilized to characterize transcriptional activity of the regulatory sequence by detecting the amount of reporter protein (i.e., α-_{L}-arabinofuranosidase) produced. Reporter gene activity can readily be detected because when the chromogenic α-_{L}-arabinofuranosidase substrate is contacted with α-_{L}-arabinofuranosidase, the substrate is converted to a colored precipitate, typically by a chemical reaction such as hydrolysis, that provides for the facile detection of reporter gene activity in a variety of environments, such as in cells and microbial colonies growing on solid agar. Advantageously, unlike β-galactosidase and other commonly used reporter genes, neither the α-_{L}-arabinofuranosidase enzyme nor its substrates are naturally present in mammalian cells, thus making the reporter gene assay of the present invention particularly suitable for use in mammalian cells. Moreover, the lack of an endogenous α-_{L}-arabinofuranosidase activity in mammalian cells simplifies detection of abfA expression by obviating the need for pretreatment of cells to eliminate background activity, as for alkaline phosphatase (2), or at a carefully buffered pH as for β-galactosidase (8).

α-_{L}-arabinofuranosidase

One component of the histochemical reporter gene assay is an abfA reporter gene. In most embodiments of the invention, the abfA reporter gene is typically operably linked to one or more regulatory sequences of interest. An abfA gene suitable for use in the present invention is one that encodes a protein having α-_{L}-arabinofuranosidase enzymatic activity. In general, proteins having α-_{L}-arabinofuranosidase enzymatic activity typically will be able to hydrolyze terminal non-reducing α-_{L}-1,2- and α-_{L}-1,3-arabinofuranosyl residues from arabinans, arabinoxylans and arabinogalactans, which are substrates predominantly present in plants. For use in the present invention, suitable abfA genes will preferably encode an α-_{L}-arabinofuranosidase that has enzymatic activity for one of the chromogenic substrates detailed below. This means when the chromogenic α-_{L}-arabinofuranosidase substrate is contacted with a suitable α-_{L}-arabinofuranosidase protein, the substrate is converted to a colored precipitate. To facilitate detection of α-_{L}-arabinofuranosidase expression and activity, preferably the enzyme is not secreted from the cell, such that it is active intracellular. While a secreted α-_{L}-arabinofuranosidase enzyme may be suitable for certain embodiments, typically if the enzyme is secreted it will be tethered to the cell membrane in accordance with methods generally known in the art.

In one embodiment, by way of non-limiting example, a suitable abfA gene is from Streptomyces livians. In a typical alternative of this embodiment, the abfA gene will have a nucleotide sequence comprising SEQ ID NO.1. In a further embodiment, a suitable abfA gene is from Bacillus subtilis. In a preferable alternative of this embodiment, the abfA gene will have a nucleotide sequence comprising SEQ ID NO 2. In still another alternative of this embodiment, the abfA gene will be from Aspergillus niger. Typically in this alternative embodiment, the abfA gene will have a nucleotide sequence comprising SEQ ID NO.3.

In still another embodiment, the isolated nucleotide sequence will encode a protein that has an amino acid sequence that is at least 50% identical to the amino acid sequence encoded by the nucleotide sequence of any of SEQ ID NOs. 1, 2 or 3. More typically, however, the isolated nucleotide sequence will encode a protein that has an amino acid sequence that is at least 75% identical to the amino acid sequence encoded by the nucleotide sequence of any of SEQ ID NOs 1, 2 or 3 and even more typically, 90% identical to the amino acid sequence encoded by the nucleotide sequence of any of SEQ ID NOs. 1, 2 or 3. In a particularly preferred embodiment, the nucleotide sequence will encode a protein that has an amino acid sequence that is at least 95%, and even more preferably, 99% identical to the amino acid sequence encoded by the nucleotide sequence of any of SEQ ID NOs. 1, 2 or 3. In each of these embodiments, the isolated nucleotide sequence will preferably encode a protein that will be able to convert the chromogenic α-_{L}-arabinofuranosidase substrate to a colored precipitate.

In certain aspects, accordingly, a nucleotide sequence that encodes a protein that is a homolog, ortholog, or degenerative variant of an α-_{L}-arabinofuranosidase is also suitable for use in the present invention. Typically, the subject proteins include fragments that share substantial sequence similarity, binding specificity and function with any of the proteins detailed above, including any of the proteins encoded by the nucleotide sequences having SEQ ID Nos. 1, 2 or 3. In each of these embodiments, the isolated nucleotide sequence will preferably encode a protein that will be able to convert the chromogenic α-_{L}-arabinofuranosidase substrate to a colored precipitate.

In determining whether a nucleotide sequence is substantially homologous or shares a certain percentage of sequence identity with an abfA nucleotide sequence suitable for use in the invention, sequence similarity may be determined by conventional algorithms, which typically allow introduction of a small number of gaps in order to achieve the best fit. In particular, “percent homology” of two proteins or two nucleic acid sequences is determined using the algorithm of Karlin and Altschul (Proc. Natl. Acad. Sci. USA 87:2264-2268, 1993). Such an algorithm is incorporated into the NBLAST and XBLAST programs of Altschul et al. (J. Mol. Biol. 215:403-410, 1990). BLAST nucleotide searches may be performed with the NBLAST program to obtain nucleotide sequences homologous to a nucleic acid molecule of the invention. Equally, BLAST protein searches may be performed with the XBLAST program to obtain amino acid sequences that are homologous to a protein of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., XBLAST and NBLAST) are employed. See http://www.ncbi.nlm.nih.gov for more details.

The invention also encompasses the use of nucleotide sequences other than a sequence that encodes a protein having an amino acid sequence selected from the group consisting of SEQ ID NO. 1, 2 or 3. Typically, these nucleotide sequences will hybridize under stringent hybridization conditions (as defined herein) to all or a portion of the nucleotide sequences described above or their complement. The hybridizing portion of the hybridizing nucleic acids is usually at least 15 (e.g., 20, 25, 30, or 50) nucleotides in length. The hybridizing portion of the hybridizing nucleic acid is at least 80%, preferably, at least 90%, and is more preferably, at least 95% identical to the sequence of a portion or all of a nucleic acid sequence encoding a protein suitable for use in the present invention, or its complement. In each of these embodiments, the isolated nucleotide sequence will preferably encode a protein that will be able to convert the chromogenic α-_{L}-arabinofuranosidase substrate to a colored precipitate.

Hybridization of the oligionucleotide probe to a nucleic acid sample is typically performed under stringent conditions. Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g., SSC or SSPE). Then, assuming at 1% mismatching results in a 1° C. decrease in the Tm, the temperature of the final wash in the hybridization reaction is reduced accordingly. For example, if sequences have greater than 95% identity with the probe is sought, the final temperature is approximately decreased by 5° C. In practice, the change in Tm can be between 0.5 and 1.5° C. per 1% mismatch. Stringent conditions involve hybridizing at 68° C. in 5×SSC/5× Denhardt's solution/1.0% SDS, and washing in 0.2×SSC/0.1% SDS at room temperature. Moderately stringent conditions include washing in 3×SSC at 42° C. The parameters of salt concentration and temperature can be varied to achieve the optimal level of identity between the probe and the subject nucleotide sequence. Additional guidance regarding such conditions is readily available in the art, for example, by Sambrook et al., 1989, Molecular Cloning, A Laboratory Manual, Cold Spring Harbor Press, N.Y.; and Ausubel et al., (eds.), 1995, Current Protocols in Molecular Biology, (John Wiley & Sons, N.Y.) at Unit 2.10.

The various nucleic acid sequences mentioned above can be obtained using a variety of different techniques known in the art. The nucleotide sequences, as well as homologous sequences encoding a suitable protein, can be isolated using standard techniques, produced recombinantly or can be purchased or obtained from a depository. Once the nucleotide sequence is obtained, it can be amplified and modified for use in a variety of applications, as further described below.

The nucleotide sequences of the present invention can be engineered using methods generally known in the art in order to alter a subject protein encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth. In certain embodiments, a signal sequence that targets the translated protein to a certain organelle within the cell is added to the nucleotide sequence. In one preferred embodiment, a nuclear signaling sequence is added to the abfA gene such that the encoded α-_{L}-arabinofuranosidase is targeted to the nucleus of the cell. For example, in order to aid in detection of α-_{L}-arabinofuranosidase activity, when used in conjunction with β-galactosidase, it is preferable to target α-_{L}-arabinofuranosidase to the nucleus when β-galactosidase is active in the cytoplasm of the same cell. Alternatively, in other embodiments it may be preferable to target β-galactosidase to the nucleus when α-_{L}-arabinofuranosidase is active in the cytoplasm.

Chromogenic α-_{L}-arabinofuranosidase Substrates

Another component of the histochemical reporter assay is a chromogenic α-_{L}-arabinofuranosidase substrate that is typically utilized to detect abfA gene activity. The chromogenic α-_{L}-arabinofuranosidase substrate facilitates detection of abfA reporter gene activity because when α-_{L}-arabinofuranosidase (i.e., the protein product of the abfA gene) contacts the substrate a colored, insoluble product is formed. One advantage of the reporter assay of the present invention, is that, depending upon the selection of the chromogenic α-_{L}-arabinofuranosidase substrate, a variety of different colored products may be formed. For example, the colored product may be blue, red or magenta. The ability to form different colored products is particularly useful to aid in the detection of reporter gene activity when the reporter assay of the present invention is employed as a part of a dual reporter gene assay within the same cell or colony. For example, when the abfA reporter gene is used in combination with the lacZ reporter gene in the same cell, detection of both genes is aided when a chromogenic substrate acted upon by α-_{L}-arabinofuranosidase forms one colored product and the chromogenic substrate acted upon by β-galactosidase forms a second contrasting colored product. By way of non-limiting example, the chromogenic substrate acted upon by α-_{L}-arabinofuranosidase may form a blue colored product and the chromogenic substrate acted upon by β-galactosidase may form a red colored product. Alternatively, the chromogenic substrate acted upon by α-_{L}-arabinofuranosidase may form a red colored product and the chromogenic substrate acted upon by β-galactosidase may form a blue colored product. Selection of chromogenic substrates in order to form a particular colored product is described in more detail below.

A number of chromogenic α-_{L}-arabinofuranosidase substrates are suitable for use in the current invention. A suitable chromogenic α-_{L}-arabinofuranosidase substrate is typically one that will form a colored, insoluble product when contacted with α-_{L}-arabinofuranosidase. In one preferred embodiment, the chromogenic substrate will comprise a furanosidase according to the following formula: 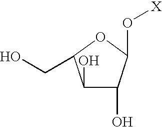
wherein:

X is a group that forms a colored product when contacted with α-_{L}-arabinofuranosidase.

In yet another preferred embodiment, the chromogenic α-_{L}-arabinofuranosidase substrate is a compound comprising an indolyl substituted with a halogen and an α-_{L}-arabinofuranoside. In an alternative of this embodiment, the chromogenic α-_{L}-arabinofuranosidase substrate is a compound comprising an indolyl substituted with a halogen and an α-_{L}-arabinofuranoside, wherein the compound is other than 5-bromo-3-indolyl-α-_{L}-arabinofuranoside.

In another alternative embodiment, the chromogenic α-_{L}-arabinofuranosidase substrate is a compound having formula (I): 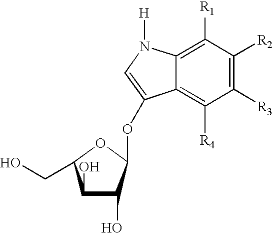
wherein:

R₁, R₂, R₃, and R₄ are independently selected from the group consisting of bromo, chloro, and hydrogen.

In an alternative embodiment for compounds having formula (I), R₁, R₂ and R₄ are independently selected from the group consisting of bromo, chloro, and hydrogen; and R₃ is chloro or hydrogen.

In another alternative embodiment for compounds having formula (I), R₁, R₂ and R₄ are hydrogen and R₃ is bromo. This compound is referred to as Z-ara. When Z-ara is contacted with α-_{L}-arabinofuranosidase, typically the colored product formed is blue. The substrate of this embodiment is a compound having the following structure:

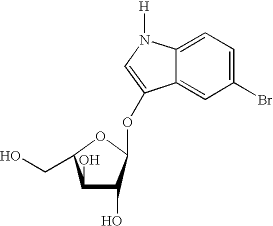

In still another alternative embodiment for compounds having formula (I), R₁ and R₂ are hydrogen, R₃ is bromo and R₄ is chloro. When the substrate of this embodiment is contacted with α-_{L}-arabinofuranosidase, typically the colored product formed is blue. The substrate of this embodiment is a compound having the following structure:

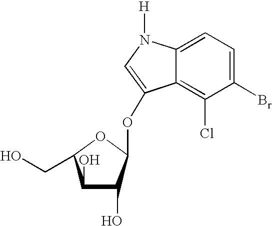

In another alternative embodiment for compounds having formula (I), R₁, R₃ and R₄ are hydrogen and R₂ is chloro. When the substrate of this embodiment is contacted with α-_{L}-arabinofuranosidase, typically the colored product formed is red or magenta. The substrate of this embodiment is a compound having the following structure:

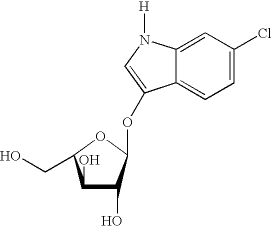

In yet another alternative embodiment for compounds having formula (I), R₁ and R₃ are hydrogen and R₂ and R₄ are chloro When the substrate of this embodiment is contacted with α-_{L}-arabinofuranosidase, typically the colored product formed is red or magenta. The substrate of this embodiment is a compound having the following structure:

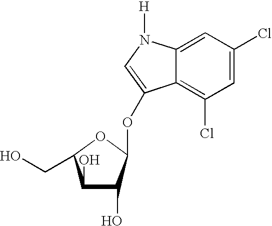

In a further alternative embodiment for compounds having formula (I), R₁ and R₂ are chloro and R₃ and R₄ are hydrogen. When the substrate of this embodiment is contacted with α-_{L}-arabinofuranosidase, typically the colored product formed is red or magenta. The substrate of this embodiment is a compound having the following structure:

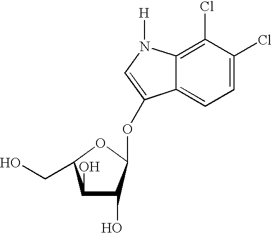

In still a further alternative embodiment for compounds having formula (I), R_{1,} R₂ and R₄ are chloro and R₃ is hydrogen. When the substrate of this embodiment is contacted with α-_{L}-arabinofuranosidase, typically the colored product formed is red or magenta. The substrate of this embodiment is a compound having the following structure:

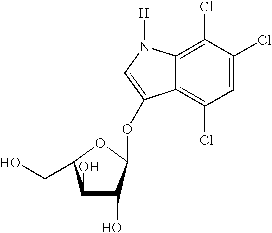

In an additional embodiment, the chromogenic substrate is a compound selected from the group consisting of 5-bromo-3-indolyl-α-_{L}-arabinofuranoside; 5-bromo-4-chloro-3-indolyl-α-_{L}-arabinofuranoside; 6-chloro-3-indolyl-α-_{L}-arabinofuranoside; 4,6-dichloro-3-indolyl-α-_{L}-arabinofuranoside; 6,7-dichloro-3-indolyl-α-_{L}-arabinofuranoside; and 4,6,7-trichloro-3-indolyl-α-_{L}-arabinofuranoside.

In still another embodiment, the chromogenic substrate is a compound selected from the group consisting of 5-bromo-4-chloro-3-indolyl-α-_{L}-arabinofuranoside; 6-chloro-3-indolyl-α-_{L}-arabinofuranoside; 4,6-dichloro-3-indolyl-α-_{L}-arabinofuranoside; 6,7-dichloro-3-indolyl-α-_{L}-arabinofuranoside; and 4,6,7-trichloro-3-indolyl-α-_{L}-arabinofuranoside.

In an additional embodiment, the chromogenic substrate is 4-methylumbelliferyl-α-_{L}-arabinofuranoside.

The chromogenic α-_{L}-arabinofuranosidase substrates of the present invention may be made by methods generally known in the art or if available, they may be purchased commercially. For example, 4-methylumbelliferyl-α-_{L}-arabinofuranoside and 4-nitrophenyl-α-_{L}-arabinofuranoside are both commercially available (Sigma-Aldrich, St. Louis, Mo., USA).

By way of non-limiting example, compounds having formula (I) may be made according to the following reaction scheme: 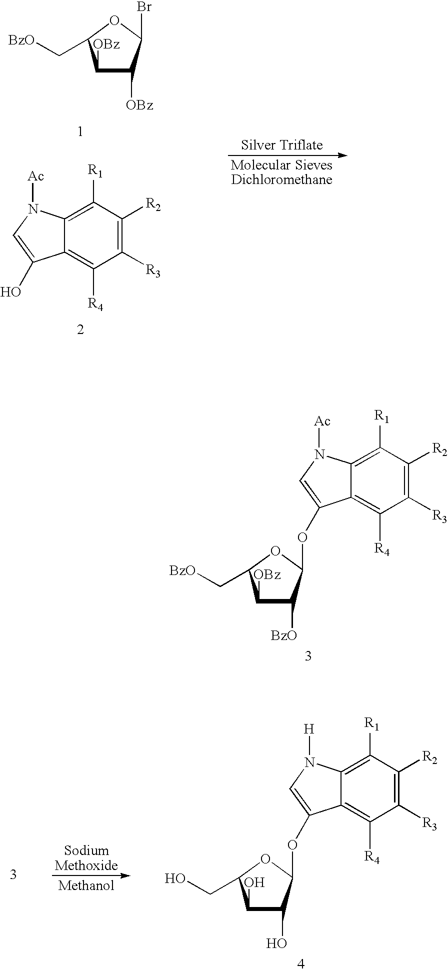
wherein:

R₁, R₂, R₃, and R₄ are independently selected from the group consisting of bromo, chloro, and hydrogen.

As detailed in the schematic diagram, the desired chromogenic substrate may be prepared by base-promoted nucleophilic substitution of the corresponding glycosyl halide (compound 1 in the schematic) with an appropriate N-acetylin-dol-3-ol (compound 2 in the schematic). In a preferred embodiment, the glycosylation process is catalyzed by silver triflate at a low reaction temperature to yield a compound having structure 3 in the schematic. The reaction is then followed by de-O-benzoylation in methanol containing a catalytic amount of sodium methoxide to yield the final product (compound 4 in schematic), which is the desired chromogenic substrate of the invention. A detailed description of the synthesis of Z-ara (i.e., 5-bromo-3-indolyl-α-_{L}-arabinofuranoside) using the illustrated method is described in Example 3.

Combinations of abfA Reporter Gene and Chromogenic α-_{L}-Arabinofuranosidase Substrate

Generally speaking, any of the abfA reporter nucleotide sequences detailed above may be combined with any of the chromogenic α-_{L}-arabinofuranosidase substrates detailed above for use in the reporter assay of the present invention. Typically, the chromogenic α-_{L}-arabinofuranosidase substrate will be selected so as to produce the desired colored product when contacted with α-_{L}-arabinofuranosidase.

In one embodiment, the abfA reporter gene is a nucleotide sequence other than SEQ ID NO. 1 and the chromogenic α-_{L}-arabinofuranosidase substrate is an indolyl substituted with a halogen and an α-_{L}-arabinofuranoside. In an alternative of this embodiment, the chromogenic α-_{L}-arabinofuranosidase substrate is a compound having formula (I): 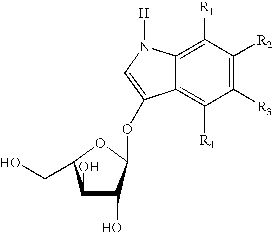
wherein:

R₁, R₂, R₃, and R₄ are independently selected from the group consisting of bromo, chloro, and hydrogen. In a further alternative of this embodiment, the chromogenic α-_{L}-arabinofuranosidase substrate is a compound selected from the group consisting of 5-bromo-3-indolyl-α-_{L}-arabinofuranoside; 5-bromo-4-chloro-3-indolyl-α-_{L}-arabinofuranoside; 6-chloro-3-indolyl-α-_{L}-arabinofuranoside; 4,6-dichloro-3-indolyl-α-_{L}-arabinofuranoside; 6,7-dichloro-3-indolyl-α-_{L}-arabinofuranoside; and 4,6,7-trichloro-3-indolyl-α-_{L}-arabinofuranoside. In each alternative of this embodiment, the abfA reporter gene may have a nucleotide sequence comprising SEQ ID No. 2 or 3.

In another embodiment, the abfA reporter gene is a nucleotide sequence comprising SEQ ID NO. 1, 2 or 3 and the chromogenic α-_{L}-arabinofuranosidase substrate is an indolyl substituted with a halogen and an α-_{L}-arabinofuranoside. In an alternative of this embodiment, the chromogenic α-_{L}-arabinofuranosidase substrate is a compound having formula (I): 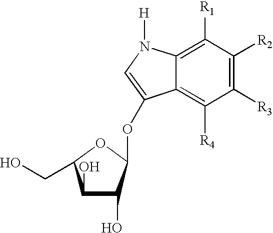
wherein:

R₁, R₂, R₃, and R₄ are independently selected from the group consisting of bromo, chloro, and hydrogen. In a further alternative embodiment, R₁, R₂ and R₄ are independently selected from the group consisting of bromo, chloro, and hydrogen; and R₃ is chloro or hydrogen. In still a further alternative of this embodiment, the chromogenic α-_{L}-arabinofuranosidase substrate is a compound selected from the group consisting of 5-bromo-3-indolyl-α-_{L}-arabinofuranoside; 5-bromo-4-chloro-3-indolyl-α-_{L}-arabinofuranoside; 6-chloro-3-indolyl-α-_{L}-arabinofuranoside; 4,6-dichloro-3-indolyl-α-_{L}-arabinofuranoside; 6,7-dichloro-3-indolyl-α-_{L}-arabinofuranoside; and 4,6,7-trichloro-3-indolyl-α-_{L}-arabinofuranoside. In each alternative of this embodiment, the abfA reporter gene may have a nucleotide sequence comprising SEQ ID No. 2 or 3.

Suitable combinations of abfA reporter nucleotide sequences and chromogenic α-_{L}-arabinofuranosidase substrates are detailed in Table A.

TABLE A

| abfA reporter nucleotide | |
|---|---|
| Sequence | Chromogenic Substrate |
| SEQ. ID NO. 1 | a compound having Formula (I) selected from |
| | the group consisting of: |
| | 5-bromo-3-indolyl-α-_{L}-arabinofuranoside; |
| | 5-bromo-4-chloro-3-indolyl-α-_{L}- |
| | arabinofuranoside; |
| | 6-chloro-3-indolyl-α-_{L}-arabinofuranoside; |
| | 4,6-dichloro-3-indolyl-α-_{L}- |
| | arabinofuranoside; |
| | 6,7-dichloro-3-indolyl-α-_{L}- |
| | arabinofuranoside; and |
| | 4,6,7-trichloro-3-indolyl-α-_{L}- |
| | arabinofuranoside |
| SEQ. ID NO. 2 | a compound having Formula (I) selected from |
| | the group consisting of: |
| | 5-bromo-3-indolyl-α-_{L}-arabinofuranoside; |
| | 5-bromo-4-chloro-3-indolyl-α-_{L}- |
| | arabinofuranoside; |
| | 6-chloro-3-indolyl-α-_{L}-arabinofuranoside; |
| | 4,6-dichloro-3-indolyl-α-_{L}- |
| | arabinofuranoside; |
| | 6,7-dichloro-3-indolyl-α-_{L}- |
| | arabinofuranoside; and |
| | 4,6,7-trichloro-3-indolyl-α-_{L}- |
| | arabinofuranoside |
| SEQ. ID NO. 3 | a compound having Formula (I) selected from |
| | the group consisting of: |
| | 5-bromo-3-indolyl-α-_{L}-arabinofuranoside; |
| | 5-bromo-4-chloro-3-indolyl-α-_{L}- |
| | arabinofuranoside; |
| | 6-chloro-3-indolyl-α-_{L}-arabinofuranoside; |
| | 4,6-dichloro-3-indolyl-α-_{L}- |
| | arabinofuranoside; |
| | 6,7-dichloro-3-indolyl-α-_{L}- |
| | arabinofuranoside; and |
| | 4,6,7-trichloro-3-indolyl-α-_{L}- |
| | arabinofuranoside |

Histochemical Reporter Gene Assay

The histochemical reporter gene assay of the present invention may be utilized in several methods to characterize a gene regulatory element of interest. In particular, the reporter gene assay provides a means to identify sequences and factors that control gene expression at the transcriptional level. For example, analysis of constructs containing various deletions within the regulatory region enables mapping of regulatory sequences necessary for transcription and cell specific expression. Other uses for the reporter gene assay of the present invention include: identification of sequences and factors that control genes at the translational level, study of mechanisms and factors that influence and alter gene expression levels and drug screening in cell-based assays.

One aspect of the invention provides a method for monitoring the transcriptional activity of a regulatory sequence of interest, such as in a cell. The cell may be a bacterial colony growing on solid agar comprising cells, an in vitro cell, such as a cell in mammalian tissue culture, or an in vivo cell, such as a cell disposed in a trangenic non human organism. In a particularly preferred embodiment, the cell is a mammalian cell. In general, the method comprises introducing into the cell a vector comprising the regulatory sequence operably linked to a nucleic acid encoding α-_{L}-arabinofuranosidase and detecting the activity of α-_{L}-arabinofuranosidase in the cell, wherein the activity of α-_{L}-arabinofuranosidase is directly proportional to the transcriptional activity of the regulatory element. Typically, the activity of α-_{L}-arabinofuranosidase is detected by contacting the cell with a sufficient amount of one of the chromogenic substrates identified above. A sufficient amount of chromogenic substrate is the amount necessary to detect α-_{L}-arabinofuranosidase activity.

The α-_{L}-arabinofuranosidase reporter assay of the invention may also be used in conjunction with other assays known in the art for detection of transcriptional activity. For example, the α-_{L}-arabinofuranosidase reporter assay may be used in combination with another assay to permit detection of the expression of two genes in a cell or organism. In a particularly preferred embodiment, the cell is a mammalian cell. A number of assays are suitable for use in combination with the reporter gene assay of the present invention. In one embodiment, the assay may be a chemiluminescent assay, such as an assay utilizing dioxetane substrates. These dioxetane substrates emit visible light following enzyme-induced degradation. Use of a chemiluminescent assay with dioxetane substrates is described in U.S. Pat. No. 5,145,772, which is incorporated herein by reference in its entirety. In another embodiment, the assay will be a reporter gene assay. A number of reporter gene assays known in the art are suitable for use in combination with the α-_{L}-arabinofuranosidase reporter assay of the current invention. By way of non-limiting example, the reporter gene assay may be chloramphenicol acetyltransferase (CAT), secreted alkaline phosphatase, luciferase, β-galactosidase, β-glucuronidase, and human growth hormone, among others.

In a preferred embodiment, the reporter gene assay will utilize β-galactosidase, the product of the lacZ gene, in combination with the reporter assay of the current invention. The method typically involves co-transfection of cells with a nucleic acid mixture of two separate vectors, each having a different reporter gene (i.e., the lacZ gene and the abfA gene) operably linked to a regulatory region of interest. The activity of α-_{L}-arabinofuranosidase and β-galactosidase is detected in the cell, wherein the activity of each enzyme is directly proportional to the transcriptional activity of the regulatory element to which the reporter gene is operably linked. Typically, the α-_{L}-arabinofuranosidase enzymatic activity is detected by contacting the cell with a sufficient amount of one of the chromogenic substrates identified above. A number of substrates are commercially available for β-galactosidase, including 5-bromo-4-chloro-3-indolyl-β-_{D}-galactosidase (i.e., X-gal) and Red-gal. A sufficient amount of chromogenic substrate is the amount necessary to detect α-_{L}-arabinofuranosidase activity or β-galactosidase, as detailed in the examples.

In order to aid in detection of α-_{L}-arabinofuranosidase activity when used in conjunction with β-galactosidase, as described above, it is preferable to target α-_{L}-arabinofuranosidase to the nucleus when β-galactosidase is active in the cytoplasm of the same cell. Alternatively, in other embodiments it may be preferable to target β-galactosidase to the nucleus when α-_{L}-arabinofuranosidase is active in the cytoplasm. Moreover, when the abfA reporter gene is used in combination with the lacZ reporter gene in the same cell, detection of both genes is aided when a chromogenic substrate acted upon by α-_{L}-arabinofuranosidase forms one colored product and the chromogenic substrate acted upon by β-galactosidase forms a second contrasting colored product. By way of non-limiting example, the chromogenic substrate acted upon by α-_{L}-arabinofuranosidase may form a blue colored product and the chromogenic substrate acted upon by β-galactosidase may form a red colored product. Alternatively, the chromogenic substrate acted upon by α-_{L}-arabinofuranosidase may form a red colored product and the chromogenic substrate acted upon by β-galactosidase may form a blue colored product. By way of non-limiting example, a composition for the detection of α-_{L}-arabinofuranosidase and β-galactoside will typically employ a chromogenic α-_{L}-arabinofuranosidase substrate selected from the group consisting of 5-bromo-4-chloro-3-indolyl-α-_{L}-arabinofuranoside; 6-chloro-3-indolyl-α-_{L}-arabinofuranoside; 4,6-dichloro-3-indolyl-α-_{L}-arabinofuranoside; 6,7-dichloro-3-indolyl-α-_{L}-arabinofuranoside; and 4,6,7-trichloro-3-indolyl-α-_{L}-arabinofuranoside; and a chromogenic β-galactosidase substrate selected from the group consisting of 5-bromo-4-chloro-3-indolyl-β-galactosidase, 6-chloroindolyl-β-galactosidase, 4,6-dichloroindolyl-β-galactosidase,6,7-dichloroindolyl-β-galactosidase, and 4,6,7-trichloroindolyl-β-galactosidase.

(A) Introduction of a Vector Comprising the Reporter Gene into Cells

In the practice of the method of the invention, one of the abfA reporter gene nucleic acids identified above is inserted into a vector and the vector is then introduced into a cell where reporter gene activity will be monitored. In embodiments where a vector comprising a second reporter gene is introduced into the cell, such as the lacZ reporter gene, the methods described herein for introduction of the abfA reporter gene into a cell may be utilized. As detailed above in certain embodiments, a signal sequence that targets the translated α-_{L}-arabinofuranosidase to a certain organelle within the cell is added to the abfA reporter gene nucleotide sequence. In one preferred embodiment, a nuclear signaling sequence is added to the abfA gene such that the encoded α-_{L}-arabinofuranosidase is targeted to the nucleus of the cell. By way of non-limiting example, synthetic oligionucleotides may be used to add the SV40 T-Ag nuclear targeting sequence to the N-terminus of α-_{L}-arabinofuranosidase.

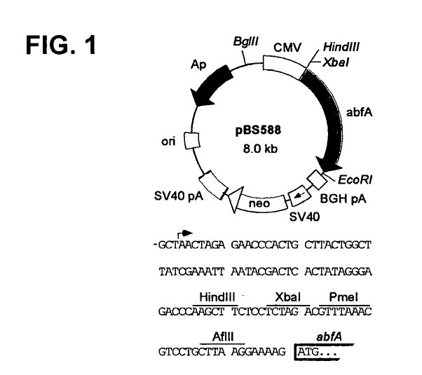

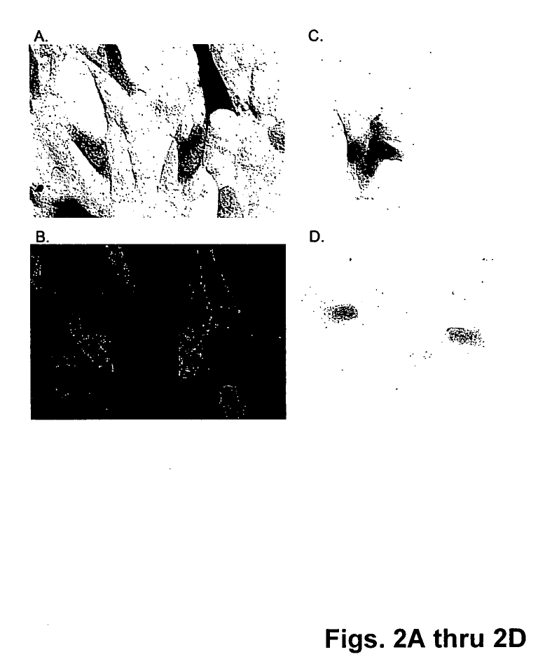

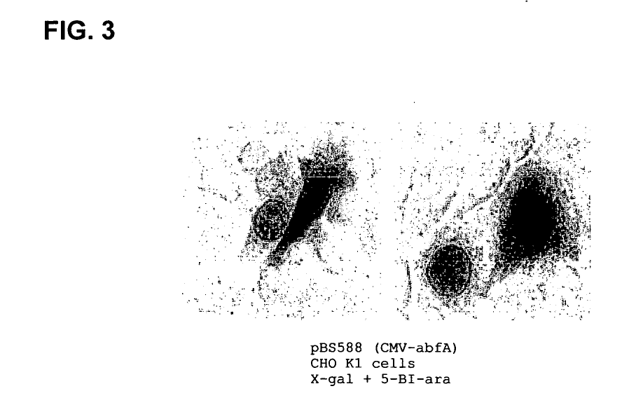

The abfA reporter gene is typically inserted into the vector such that it is operably linked to the regulatory sequence of interest. A number of vectors are suitable for use in the present invention. In one preferred embodiment, the vector is the CMV abfA expression vector detailed FIG. 1. Referring to FIG. 1, the Streptomyces lividans abfA gene is placed under the control of the human cytomegalovirus immediate early promoter (hCMV) with the bovine growth hormone polyadenylation signal (BGH pA) by cloning into pcDNA3 (Invitrogen). While the vector detailed in FIG. 1 contains the Streptomyces lividans abfA gene, the vector contains several unique restriction sites to facilitate subcloning of another abfA gene detailed herein. Moreover, the hCMV promoter can be replaced with a regulatory sequence of interest by methods generally known in the art.

A number of vectors in addition to the vector detailed in FIG. 1 are suitable for use in the practice of the current invention. Depending upon the embodiment, either eukaryotic or prokaryotic vectors may be used. Suitable eukaryotic vectors that may be used include MSCV, Harvey murine sarcoma virus, pFastBac, pFastBac HT, pFastBac DUAL, pSFV, pTet-Splice, pEUK-C1, pPUR, pMAM, pMAMneo, pBI101, pBI121, pDR2, pCMVEBNA, YACneo, pSVK3, pSVL, pMSG, pCH110, pKK232-8, p3'SS, pBlueBacIII, pCDM8, pcDNA1, pZeoSV, pcDNA3, pREP4, pCEP4, and pEBVHis vectors. Suitable prokaryotic vectors that can be used in the present invention include pET, pET28, pcDNA3.1/V5-His-TOPO, pCS2+, pcDNA II, pSL301, pSE280, pSE380, pSE420, pTrcHis, pRSET, pGEMEX-1, pGEMEX-2, pTrc99A, pKK223-3, pGEX, pEZZ18, pRIT2T, pMC1871, pKK233-2, pKK38801, and pProEx-HT vectors.

Methods that are well known to those skilled in the art may be used to construct expression vectors containing an abfA reporter gene operably linked to a regulatory sequence of interest. These methods include, for example, in vitro recombinant DNA techniques, synthetic techniques, and in vivo genetic recombination. (See, e.g., Sambrook, J. et al. (1989) Molecular Cloning, A Laboratory Manual, Cold Spring Harbor Press, Plainview N.Y., ch. 4, 8, and 16-17; Ausubel, F. M. et al. (1995) Current Protocols in Molecular Biology, John Wiley & Sons, New York N.Y., ch. 9, 13, and 16).

In one embodiment, a site-specific recombinase system is employed to allow for the reporter gene to be conditionally expressed. The site-specific recombinase system may include those generally known in the art, including site-specific DNA recombinases, such as either the yeast-derived Flp/frt, or the bacteriophage-derived Cre/loxP system. By way of example, the Cre/loxP recombination system may be employed by constructing a vector that has the regulatory region of interest flanked by two loxP sites, where the loxP sites typically have intervening DNA sequence disposed in between. The regulatory region is also placed within the vector so that it is operably linked to the reporter gene only when the loxP sites are contacted with Cre. In this embodiment, reporter gene expression occurs only when Cre is contacted with the loxP sites, thus operably linking the regulatory region of interest to the reporter gene.

The vector comprising the abfA gene operably linked to a regulatory sequence of interest can be introduced into a target cell by mechanical, electrical or chemical procedures. Mechanical methods include microinjection, pressure, and particle bombardment. Electrical methods include electroporation. Chemical methods include liposomes, DEAE-dextran, calcium phosphate, artificial lipids, proteins, dendrimers, or other polymers, including controlled-release polymers.

In one aspect of this embodiment, accordingly, a mechanical method is employed to introduce the subject vector into the target cell. One such method is hydrodynamic force and other external pressure-mediated DNA transfection methods. Alternatively, ultrasonic nebulization can be utilized for DNA-lipid complex delivery. In other suitable embodiments, particle bombardment, also known as biolistical particle delivery, can be utilized to introduce DNA into several cells simultaneously. In still another alternative mechanical method, DNA-coated microparticles (e.g., gold, tungsten) are accelerated to high velocity to penetrate cell membranes or cell walls. This procedure is used predominantly in vitro for adherent cell culture transfection.

In a further aspect of this embodiment, an electrical method is employed to introduce subject nucleotide sequences into the target cell. In one alternative of this embodiment, electroporation is employed. Electroporation uses high-voltage electrical impulses to transiently permeabilize cell membranes, and thereby, permits cellular uptake of macromolecules, such as nucleic acid and protein sequences.

In an additional aspect of this embodiment, a chemical method is employed to introduce a selected nucleotide sequences into the target cell. Chemical methods, using uptake-enhancing chemicals, are highly effective for delivering nucleic acids across cell membranes. For example, nucleotide sequences are typically negatively charged molecules. DEAE-dextran and calcium phosphate, which are positively charged molecules, interact with nucleotide sequences to form DEAE-dextran-DNA and calcium phosphate-DNA complexes, respectively. These complexes are subsequently internalized into the target cell by endocytosis.

In another alternative embodiment, the chemical enhancer is lipofectin-DNA. This complex comprises an artificial lipid-based DNA delivery system. In this embodiment, liposomes (either cationic, anionic, or neutral) are complexed with DNA. The liposomes can be used to enclose a subject nucleic acid for delivery to target cells, in part, because of increased transfection efficiency.

In yet another alternative chemical embodiment, protein-based methods for DNA introduction may also utilized. The cationic peptide poly-L-lysine (PLL) can condense DNA for more efficient uptake by cells. Protamine sulfate, polyamidoamine dendrimers, synthetic polymers, and pyridinium surfactants may also be utilized.

In still a further chemical embodiment for nucleotide introduction, biocompatible controlled-release polymers may be employed. Biodegradable poly (D,L-lactide-co-glycolide) microparticles and PLGA microspheres have been used for long-term controlled release of DNA molecules to cells. In a further embodiment, the subject nucleotide sequences may also be encapsulated into poly(ethylene-co-vinyl acetate) matrices, resulting in long term controlled, predictable release for several months.

(b) Detection of Reporter Gene Activity

After introducing either a vector having an abfA gene operably linked to a regulatory sequence of interest into the cell or co-introducing a vector having an abfA gene operably linked to a regulatory sequence of interest and a vector having a lacZ gene operably linked to a regulatory sequence of interest, enzymatic activity is detected. A number of methods are suitable for detecting reporter enzymatic activity including: isotopic, calorimetric, fluorometric or luminescent enzyme substrates and immunoassay-based procedures with isotopic or color endpoints.

In a preferred embodiment, one of the chromogenic α-_{L}-arabinofuranosidase substrates detailed above is utilized to detect α-_{L}-arabinofuranosidase. In this procedure, the cell is contacted with a sufficient amount of the chromogenic substrate and the enzymatic activity of α-_{L}-arabinofuranosidase is detected based upon the amount of colored precipitate formed. The amount of colored precipitate formed may be determined by methods generally known in the art, such as by the in situ α-arabinofuranosidase assay detailed in the Examples. Any of the chromogenic substrates detailed herein may be utilized to detect β-galactosidase activity in the same manner as detailed for detection of α-_{L}-arabinofuranosidase activity. The amount of colored precipitate formed when β-galactosidase is contacted with a chromogenic substrate may be determined by lo methods generally known in the art, such as by the in situ β-galactosidase assay detailed in the Examples.

Immunohistochemical Detection

A further aspect of the invention encompasses the use of the chromogenic substrates of the invention in conjunction with α-_{L}-arabinofuranosidase as an enzyme label in immunological diagnostic and immunohistochemical reactions. Typically, the α-_{L}-arabinofuranosidase is conjugated to a primary or secondary antibody to produce α-_{L}-arabinofuranosidase-labeled antibodies. The enzyme-labeled antibodies are then utilized in methods to localize a specific antigen in a tissue sample. In this manner, the immunohistochemistry method of the invention may be utilized, for example, to determine the localization of antigens in tissue sections by the use of α-_{L}-arabinofuranosidase-labeled antibodies as specific reagents through antigen-antibody interactions that are visualized by chromogenic substrate of the invention.

The immunohistochemical method may be employed in clinical diagnostics to determine, for example, the localization of antigens in tissue sections. In immunoassay procedures typically used in clinical diagnostics, antibodies and antigens are conjugated with α-_{L}-arabinofuranosidase, forming enzyme conjugates, to detect the presence and/or amount of various analytes, i.e., antigens or antibodies in test samples prepared from biological fluids such as plasma, serum, spinal fluid or amniotic fluid. The test samples may be collected and contacted with the α-_{L}-arabinofuranosidase-labeled antibodies by methods generally know in the art. The presence and/or the amount of the analyte can be determined by measuring the formation of the resulting antibody-antigen-enzyme complexes. The antibody-antigen-enzyme complexes may be detected by measuring the degree of colorimetric change resulting from contacting the chromogenic substrate of the invention with the α-_{L}-arabinofuranosidase-labeled antibodies. The colorimetric change as detailed herein, can be determined instrumentally by measuring the absorbance of the solution, or in some cases, visually, to provide an indication of the analyte. In addition to the use of enzyme conjugated antibodies to detect antigens, the method can also be used to detect the presence of antibodies by reversing the roles of antigens and antibodies in the foregoing procedure.

The antibody may be conjugated to α-_{L}-arabinofuranosidase by methods generally known in the art. Since only a relatively small amount of enzyme conjugate is needed for most immunoassays, the enzyme conjugate composition will usually be present in an aqueous solution of buffer or the like. In certain embodiments, the amount of enzyme conjugate in the buffer is usually no more than about 1 microgram per milliliter (ug/ml). In other embodiments, however, at least 0.01 ug/ml of the enzyme conjugate is usually present in the solution. The identity of such a buffer is not critical to the present invention, and suitable buffers can be selected from a variety of aqueous solutions known in the art, such as saline solutions, borate solutions and other common buffers, such as phosphate and citrate. When the aqueous solution is a saline solution, it is generally preferred that it comprise about 0.1% to 2% sodium chloride.

Cells and Transeenic Non Human Organisms

Another aspect of the invention provides a cell or a cell disposed within a transgenic non-human organism that comprises an abfA reporter gene either alone or in addition to an α-_{L}-arabinofuranosidase chromogenic substrate. In a particularly preferred embodiment, the cell is a mammalian cell. The cell may contain any of the combinations of abfA reporter genes and chromogenic substrates described herein. Alternatively, the cell may comprise an abfA reporter gene, a lacZ reporter gene, an α-_{L}-arabinofuranosidase chromogenic substrate, and a β-galactosidase chromogenic substrate in accordance with any of the combinations detailed herein. The genes and chromogenic substrates may be introduced into a cell in accordance with methods generally known in the art, such as by the methods described herein.

Procedure for producing a transgenic organisms are known in the art; for example, see B. Hogan et al., “Manipulating the Mouse Embryo: A Laboratory Manual”, Cold Spring Harbor Laboratory, Cold Spring Harbor, N.Y., USA, 1986 and T. E. Wagner and P. C. Hoppe, U.S. Pat. No. 4,873,191, issued Oct. 10, 1989. Included within the scope of the invention is a method of producing a transgenic organism, carrying the abfA transgene (i.e., reporter gene), that can be stably bred to produce offspring containing the abfA reporter gene. In general, the method comprises:
(a) isolating a fertilized oocyte from a first female organism;
(b) transferring the abfA reporter gene into the fertilized oocyte;
(c) transferring the fertilized oocyte containing the abfA reporter gene to the uterus of the same species as the first organism;
(d) maintaining the second female organism such that
   (i) the second female organism becomes pregnant with the embryo derived from the fertilized oocyte containing the abfA reporter gene,
   (ii) the embryo develops into the transgenic organism, and
   (iii) the transgenic organism is viably born from the second female organism;
wherein the transgenic organism has the genetic sequence for the abfA reporter gene and is capable of being bred to produce offspring having cells stably containing the genetic sequence.

In an exemplary embodiment an abfA transgenic mouse may be produced by methods that are generally known in the art, such as by using homologous recombination in embryonic stem cells (See, e.g., U.S. Pat. No. 5,175,383 and U.S. Pat. No. 5,767,337.). For example mouse embryonic stem (ES) cells, such as the mouse 129/SvJ cell line, are derived from the early mouse embryo and grown in culture. In one embodiment, homologous recombination takes place using the Cre/loxP system to knock-out a gene of interest in a tissue- or developmental stage-specific manner, as known in the art (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Alternatively, when utilizing a knock-in method, polynucleotides encoding a target DNA segment can be used to create transgenic animals (mice or rats). Typically, a region of a polynucleotide encoding a target DNA segment is injected into animal embryonic stem cells, and the injected sequence integrates into the animal cell genome. Transformed cells are injected into blastulae, and the blastulae are implanted as described above.

Kits

A further aspect of the invention provides a kit for monitoring the transcriptional activity of a regulatory sequence of interest. In one embodiment, the kit will have a vector comprising an insertion site for the regulatory element operably linked to a nucleic acid encoding α-_{L}-arabinofuranosidase, a chromogenic α-_{L}-arabinofuranosidase substrate, and instructions for detecting the activity of α-_{L}-arabinofuranosidase. Any combination of nucleic acids encoding α-_{L}-arabinofuranosidase and α-_{L}-arabinofuranosidase chromogenic substrates detailed herein may be utilized in the kit.

Alternatively, the invention provides a kit for monitoring the transcriptional activity of two regulatory sequences of interest. Typically for this embodiment, the kit will have a vector comprising an insertion site for a first regulatory element operably linked to a nucleic acid encoding α-_{L}-arabinofuranosidase, a vector comprising an insertion site for a second regulatory element operably linked to a nucleic acid encoding β-galactosidase, a chromogenic α-_{L}-arabinofuranosidase substrate, a chromogenic β-galactosidase substrate and instructions for detecting the activity of α-_{L}-arabinofuranosidase and β-galactosidase. Any combination of nucleic acids encoding α-_{L}-arabinofuranosidase and β-galactosidase may be utilized in the kit. In addition any combination of β-galactosidase chromogenic substrates and α-_{L}-arabinofuranosidase chromogenic substrates detailed herein may be utilized in the kit.

All publications, patents, patent applications and other references cited in this application are herein incorporated by reference in their entirety as if each individual publication, patent, patent application or other reference were specifically and individually indicated to be incorporated by reference.

### DEFINITIONS

abfA stands for a gene that encodes α-_{L}-arabinofuranosidase.

Cell as used herein refers to either a prokaryotic cell or an eukaryotic cell. Examples of such cells include bacterial cells, yeast cells, mammalian cells, plant cells, insect cells or fungal cells.

Conservative amino acid substitutions are those substitutions that do not abolish the ability of a subject protein to participate in the biological functions as described herein. Typically, a conservative substitution will involve a replacement of one amino acid residue with a different residue having similar biochemical characteristics such as size, charge, and polarity versus non polarity. A skilled artisan can readily determine such conservative amino acid substitutions.

DNA segment refers to a linear fragment of single- or double-stranded deoxyribonucleic acid (DNA), which can be derived from any source.

The term expression as used herein is intended to mean the synthesis of gene product from a gene coding for the sequence of the gene product. The gene product can be RNA or a protein.

A gene is a hereditary unit that has one or more specific effects upon the phenotype of the organism that can mutate to various allelic forms.

Homology describes the degree of similarity in nucleotide or protein sequences between individuals of the same species or among different species. As the term is employed herein, such as when referring to the homology between either two proteins or two nucleotide sequences, homology refers to molecules having substantially the same function, but differing in sequence. Most typically, the two homologous molecules will share substantially the same sequence, particularly in conserved regions, and will have sequence differences in regions of the sequence that does not impact function.

A host organism is an organism that receives a foreign biological molecule, including an antibody or genetic construct, such as a vector containing a gene. The organism may be either a prokaryote or an eukaryote. For example, the organism may be a bacteria, a yeast, a mammal, a plant, an insect, or a fungus.

Knock-in, as used herein, is commonly understood to be the placement into the genome by homologous recombination of a transgene at a specific locus such that it is under the regulatory control of genetic elements endogenous to that locus. In a typical embodiment, a knock-in procedure will be used to substitute the transgene for an endogenous gene in the genome of the transgenic organism.

Knock-out, as used herein, is commonly understood to be the placement into the genome by homologous recombination of a transgene at a specific locus such that placement of the transgene results in the ablation of an endogenous gene at the specific locus.

A nucleic acid is a nucleotide polymer better known as one of the monomeric units from which DNA or RNA polymers are constructed, it consists of a purine or pyrimidine base, a pentose, and a phosphoric acid group.

Peptide is defined as a compound formed of two or more amino acids, with an amino acid defined according to standard definitions, such as is found in the book “A Dictionary of Genetics” by King and Stansfield.

Plasmids are double-stranded, closed DNA molecules ranging in size from 1 to 200 kilo-bases. Plasmids are incorporated into vectors for transfecting a host with a nucleic acid molecule.

A polypeptide is a polymer made up of less than 350 amino acids.

Protein is defined as a molecule composed of one or more polypeptide chains, each composed of a linear chain of amino acids covalently linked by peptide bonds. Most proteins have a mass between 10 and 100 kilodaltons. A protein is often symbolized by its mass in kDa.

Polyadenylation nucleotide sequence or polyadenylation nucleotide region refers to a nucleotide sequence usually located 3′ to a coding region which controls the addition of polyadenylic acid to the RNA transcribed from the coding region in conjunction with the gene expression apparatus of the cell.

As used herein, the term promoter region refers to a sequence of DNA, usually upstream (5′) of the coding sequence, which controls the expression of the coding region by providing the recognition for RNA polymerase and/or other factors required for transcription to start at the correct site. A “promoter fragment” constitutes a DNA sequence consisting of the promoter region. A promoter region can include one or more regions that control the effectiveness of transcription initiation in response to physiological conditions, and a transcription initiation sequence.

Regulatory nucleotide sequence as used herein, refers to a nucleotide sequence located proximate to a coding region whose transcription is controlled by the regulatory nucleotide sequence in conjunction with the gene expression apparatus of the cell. Generally, the regulatory nucleotide sequence is located 5′ to the coding region. A promoter can include one or more regulatory nucleotide sequences.

A vector is a self-replication DNA molecule that transfers a DNA segment to a host cell.

Z-ara is 5-bromo-3-indolyl-α-arabinofuranoside.

As various changes could be made in the above compounds, products and methods without departing from the scope of the invention, it is intended that all matter contained in the above description and in the examples given below, shall be interpreted as illustrative and not in a limiting sense.

### EXAMPLES

Examples 1 and 2 demonstrate the use of the histochemical reporter assay of the invention either alone for detection of α-_{L}-arabinofuranosidase activity in a cell, and as a part of a dual detection system to detect α-_{L}-arabinofuranosidase activity and β-galactosidase activity in the same cell.

### Example 1

To express α-_{L}-arabinofuranosidase in mammalian cells the S. lividans abfA gene was placed under the control of the strong human cytomegalovirus (hCMV) major immediate early promoter by cloning the gene into pcDNA3 (Invitrogen, La Jolla, Calif., USA) to give CMV-abfA. FIG. 1 depicts a schematic of the CMV abfA expression vector. Briefly, the S. lividans abfA gene from plasmid pBS460 (4) was placed under the control of the human cytomegalovirus immediate early promoter (hCMV) with the bovine growth hormone polyadenylation signal (BGH pA) by cloning into pcDNA3 to give the CMV-abfA vector. NIH 3T3 cells were transfected with CMV-abfA and 30 hours later were fixed with 2% formaldehyde, 0.2% glutaraldehyde for 5 minutes and then processed for histochemical detection of abfA expression using a standard protocol for lacZ detection (5), but substituting Z-ara (0.02%) for X-gal. The synthesis for Z-ara is described in Example 3. Just like X-gal, Z-ara is a colorless water-soluable chromogen that forms an insoluable blue precipitate upon hydrolysis. Cells expressing abfA are dark blue on Z-ara, but show no color change on X-gal. Conversely, cells expressing lacZ remain colorless on Z-ara. Alternative β-galactosidase substrates to X-gal, 6-chloro-3-indolyl-β-D-galactopyranaside (“Red-gal,” Research Organics, Cleveland, Ohio, USA) and 5-bromo-6-chloro-3-indolyl-β-D-galactopyranoside (“Magenta-gal,” BioSynth AG, Switzerland), allow LacZ⁺ cells to appear red instead of blue and thus easily distinguishable from Z-ara positive cells.

FIG. 2(A) shows a representative field of cells from such an experiment with abfA-expressing cells stained blue and non-transfected cells white. As expected, blue staining is predominantly cytoplasmic since α-_{L}-arabinofuranosidase is a hexamer of 60 kDa subunits and thus too large to efficiently enter the nucleus without a nuclear localization signal (6). A similar level of blue staining was obtained with Z-ara after a 48 hour expression period and, as expected, no blue staining was observed when X-gal (US Biological, Swampscott, Mass., USA) was substituted for Z-ara in the staining procedure. One feature of the Z-ara substrate is that the 5-bromo-3-indoloxyl-derived final product has birefringent properties. FIG. 2(B) shows that under polarized light Z-ara stained AbfA⁺ cells exhibit a distinct yellow birefringence. Even cells showing only light blue staining were easily detected under polarized light, although intensely stained blue regions appeared opaque to detection by birefringence.

### Example 2

To determine whether α-_{L}-arabinofuranosidase and β-galactosidase could be detected both in the same cell, NHI 3T3 cells were co-transfected with CMV-abfA and with proBGN (7), a plasmid encoding the lacZ gene tagged with a nuclear localization signal. The NIH 3T3 cells were grown on a glass cover slip and then transfected with 1.5 μg plasmid DNA per well of a 6-well dish using Polyfect (Qiagen) as suggested by the manufacturer. One day after transfection cells were fixed followed by in situ visualization of β-galactosidase and α-_{L}-arabinofuranosidase activities by incubation with 0.02% Magenta-gal and 0.02% Z-ara, respectively. Imaging was with a Leica DMR microscope mounted with an Optronics Magnafire digital camera. FIGS. 2(C) and 2(D) show that co-transfected cells exhibited a red nucleus (from lacZ expression) surrounding a blue cytoplasm (from abfA expression). Thus, two distinct reporter genes, lacZ and abfA, were histochemically detected and distinguished in the same mammalian cell at the same time using the same chemistry and visualization procedure.

### Example 3

To synthesize Z-ara, the following reaction scheme was employed:

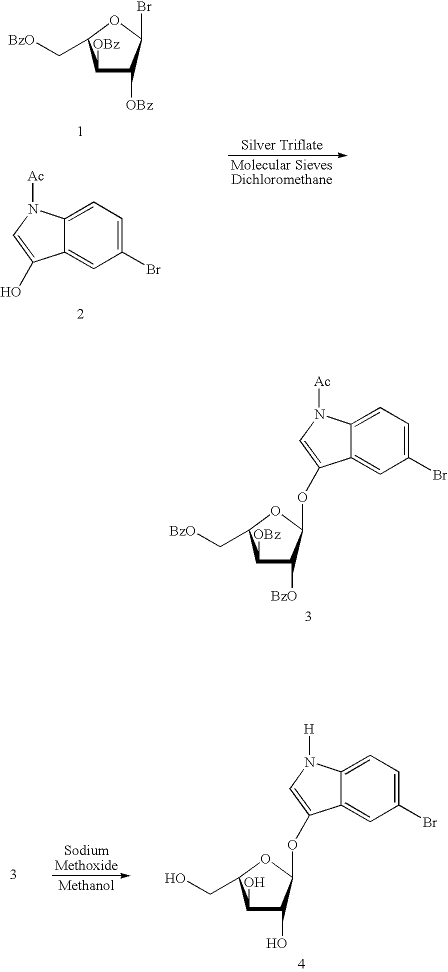

2,3,5-Tri-O-benzoyl-α-_{L}-arabinofuranosyl bromide (1). Following the method of Ness and Fletcher (6), a mixture of 2 g L-arabinose and 2 g Dowex AG 50-X-8 (Bio-Rad, Richmond, Calif.) in methanol was stirred at room temperature overnight (or 2 h at the reflux temperature) to generate the methyl furanosides. After filtration of the resin, the solution was evaporated and residual methanol was removed by azeotropic distillation with pyridine in vacuo. A portion of the resulting, syrupy mixture of glycosides (1.09 g) was directly benzoylated in the usual manner with 3.5 eq benzoyl chloride (2.7 ml) in pyridine (15 ml). The product was isolated in about 60% yield by crystallization from methanol. A portion of this product (0.5 g) was dissolved in 2 ml glacial acetic acid and treated, at room temperature, for about 30 min with 1 ml of hydrogen bromide-saturated glacial acetic acid. The solution was diluted with dichloromethane and washed with ice water and then ice-saturated sodium hydrogen carbonate solution. After drying of the dichloromethane solution over anhydrous magnesium sulfate, the product (a white foam, which is very difficult to crystallize, obtained after removal of the solvent in vacuo) was used directly for preparation of the indole glycoside. The ¹H NMR spectrum of the material matched that reported by Bock and Pedersen (7).

1-Acetyl-5-bromo-indoxyl-3-ol (2). For the preparation of the intermediate, 1-acetyl-5-bromo-3-indolyl acetate (8), 0.5 g 5-bromoindoxyl acetate (Research Organics, Cleveland, Ohio) was dissolved in 2 ml acetic anhydride, treated with 0.2 μl triethylamine, and heated for 2-4 h at 60° C. After cooling, the solution was poured into strongly stirred ice water. After 20 min, the crystalline product was isolated by filtration and was recyrstallized from ethanol. The bis-acetate was de-O-acetylated in acid as described for the preparation of 1-acetyl-5-bromo-6-chloroindol-3-ol (9). The product, 2, was conveniently recrystallized from acetone as colorless to pale-blue needles.

1-Acetyl-5-bromo-3-indolyl 2, 3, 5-tri-O-benzoyl-α-_{L}-arabinofuranoside (3). Compounds 1 (0.55 g, 1.05 mmol) and 2 (0.3 g, 1.2 mmol) were dissolved in 20 ml of dry dichloromethane and degassed with a stream of nitrogen gas. Powdered 4-Å molecular sieves (0.5 g) were added and the mixture, which was kept protected from light, was cooled at 0° C. Silver triflate (0.31 g, 1.2 mmol) was added in one portion and the solution was allowed to warm to room temperature for 30 min. After a total of about 2 h, the solution was filtered, the solids were washed with dichloromethane, and the filtrate was evaporated. The product was applied to a column of silica gel (50 g) with hexane:ethyl acetate (3:2) as the eluant. Appropriate fractions (determined by thin-layer chromatography on silica gel plates) were pooled and evaporated, yielding 3 as a white foam in about 60% yield. The ¹H NMR spectrum (data provided below) was fully consistent with the data reported for α-_{L}-arabinofuranosides, based on the pattern of the scaler coupling constants (10). ¹H NMR for 3: 7.95-8.18 and 7.25-7.68 ppm, m, 15 H, benzoyl; 5.84 ppm, d, 1H, H-1, J_{1,2}<0.5 Hz; 5.76 ppm, s, 1H, H-2, J_{2,3}<1 Hz; 5.65 ppm, s, 1H, H-3, J_{3,4}=4 Hz; 4.66-4.80, m, 3 H, H-4, H-5,5′.

5-Bromo-3-indolyl α-_{L}-arabinofuranoside (4). Compound 3 was treated in the conventional manner with anhydrous methanol containing a small amount of sodium methoxide for about 10 h at 25° C. The reaction was stopped by the addition of a few drops of glacial acetic acid and the solvent was removed in vacuo. The syrupy 4 was isolated by column chromatography on silica gel with chloroform:methanol (3:1) as the eluant in about 8% yield.

As illustrated in Examples 1 and 2, the abfA gene and its use with a chromogenic substrate provide an additional histochemical detection system to the molecular toolbox for monitoring expression of a gene in mammalian systems. The detection compatibility of α-_{L}-arabinofuranosidase with β-galactosidase demonstrates that the abfA gene can be used in conjunction with a lacZ reporter gene to monitor two overlapping patterns of gene expression in mammalian cells.

### REFERENCES

All references cited in the preceding text of the patent application or in the following reference list, to the extent that they provide exemplary, procedural, or other details supplementary to those set forth herein, are specifically incorporated by reference to the same extent as if each individual publication or patent application was specifically and individually indicated to be incorporated by reference.
1. Manin, C., F. Shareek, R. Morosoli and D. Kluepfel. 1994. Purification and characterization of an α-_{L}-arabinofuranosidase from Streptomyces lividans 66 and DNA sequence of the gene (abfA). Biochem. J. 302:443-449.
2. DePrimo, S. E., J. Coo, M. N. Hersh and J. R. Stringer. 1998. Use of human placental alkaline phosphatase transgenes to detect somatic mutation in mice in situ. Methods 16:49-61.
3. Kaji, A. 1956. L-arabinosidases. Adv. Carbohydr. Chem. Biochem. 42:383-394.
4. Berlin, W. and B. Sauer. 1996. In situ color detection of α-_{L}-arabinofuranosidase, a “no-background” reporter gene, with 5-bromo-3-indolyl-α-_{L}-arabinofuranoside. Anal. Biochem. 243; 171-175.
5. Sanes, J. R., J. L. R. Rubenstein and J.-F. Nicolas. 1986. Use of a recombinant retrovirus to study post-implantation cell lineage in mouse embryos. EMBO. J. 5:3133-3142.
6. Dingwall, C. and R. A. Laskey. 1991. Nuclear targeting sequences-a consensus? TIBS 16:478-481.
7. Manrow, R. E., A. R. Sburlati, J. A. Hanover and S. L. Berger. 1991. Nuclear targeting of prothymosin alpha. J. Biol. Chern. 266:3916-3924.
8. Hendrikx, P. J., A. C. Martens, J. W. Visser and A. Hagenbeek. 1994. Differential suppression of background mammalian lysosomai beta-galactosidase increases the detection sensitivity of LacZ-marked leukemic cells. Anal. Biochem. 222:456-460.
9. Aguzzi, A. and F. Theuring. 1994. Improved in situ beta-galactosidase staining for histological analysis of transgenic mice. Histochem. 102:477-481.
10. Baron, U. and H. Bujard. 2000. Tet repressor-based system for regulated gene expression in eukaryotic cells: principles and advances. Methods Enzynnol 327:401-421.
11. Sauer, B. 2002. Cre/lox: one more step in the taming of the genome. Endocrine 19:221-228.
12. Lobe, C. G., K. E. Koop, W. Kreppner, H. Lomeli, M. Gertsenstein and A. Nagy. 1999. Z/AP, a double reporter for cre-mediated recombination. Dev. Biol. 208:281-292.

What is claimed is:

1. A reporter gene assay, the assay comprising:
(a) an isolated nucleic acid encoding an α-_{L}-arabinofuranosidase, the nucleic acid being other than a nucleic acid from Streptomyces livians; and
(b) a chromogenic α-_{L}-arabinofuranosidase substrate.

2. The reporter gene assay of claim 1, wherein the isolated α-_{L}-arabinofuranosidase nucleic acid is from Bacillus subtilis or Aspergillus niger.

3. The reporter gene assay of claim 1, wherein the chromogenic α-_{L}-arabinofuranosidase substrate comprises an indolyl substituted with a halogen and an α-_{L}-arabinofuranoside.

4. The reporter gene assay of claim 1, wherein the chromogenic α-_{L}-arabinofuranosidase substrate is a compound having the formula: 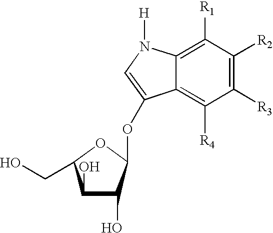
wherein:
R₁, R₂, R₃, and R₄ are independently selected from the group consisting of bromo, chloro, and hydrogen.

5. The reporter gene assay of claim 1, wherein the chromogenic α-_{L}-arabinofuranosidase substrate is a compound selected from the group consisting of 5-bromo-3-indolyl-α-_{L}-arabinofuranoside; 5-bromo-4-chloro-3-indolyl-α-_{L}-arabinofuranoside; 6-chloro-3-indolyl-α-_{L}-arabinofuranoside; 4,6-dichloro-3-indolyl-α-_{L}-arabinofuranoside; 6,7-dichloro-3-indolyl-α-_{L}-arabinofuranoside; and 4,6,7-trichloro-3-indolyl-α-_{L}-arabinofuranoside.

6. A reporter gene assay, the assay comprising:
(a) an isolated nucleic acid encoding an α-_{L}-arabinofuranosidase; and
(b) a chromogenic α-_{L}-arabinofuranosidase substrate having formula (I): 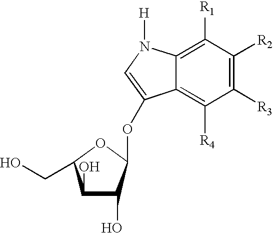
wherein:
R₁, R₂ and R₄ are independently selected from the group consisting of bromo, chloro, and hydrogen; and
R₃ is chloro or hydrogen.

7. The reporter gene assay of claim 6, wherein the isolated α-_{L}-arabinofuranosidase nucleic acid is from Bacillus subtilis or Aspergillus niger.

8. The reporter gene assay of claim 6, wherein the chromogenic α-_{L}-arabinofuranosidase substrate is a compound selected from the group consisting of 5-bromo-4-chloro-3-indolyl-α-_{L}-arabinofuranoside; 6-chloro-3-indolyl-α-_{L}-arabinofuranoside; 4,6-dichloro-3-indolyl-α-_{L}-arabinofuranoside; 6,7-dichloro-3-indolyl-α-_{L}-arabinofuranoside; and 4,6,7-trichloro-3-indolyl-α-_{L}-arabinofuranoside.

9. A reporter gene assay, the assay comprising:
(a) -an isolated nucleic acid encoding an α-_{L}-arabinofuranosidase from Bacillus subtilis or Aspergillus niger; and
(b) a chromogenic α-_{L}-arabinofuranosidase substrate.

10. The reporter gene assay of claim 9, wherein the chromogenic α-_{L}-arabinofuranosidase substrate comprises an indolyl substituted with a halogen and an α-_{L}-arabinofuranoside.

11. The reporter gene assay of claim 9, wherein the chromogenic α-_{L}-arabinofuranosidase substrate is a compound having the formula: 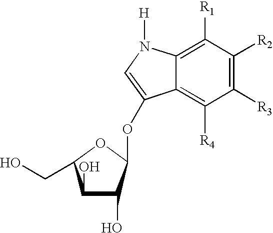
wherein:
R₁, R₂, R₃, and R₄ are independently selected from the group consisting of bromo, chloro, and hydrogen.

12. The reporter gene assay of claim 9, wherein the-chromogenic α-_{L}-arabinofuranosidase substrate is a compound selected from the group consisting of 5-bromo-3-indolyl-α-_{L}-arabinofuranoside; 5-bromo-4-chloro-3-indolyl-α-_{L}-arabinofuranoside; 6-chloro-3-indolyl-α-_{L}-arabinofuranoside; 4,6-dichloro-3-indolyl-α-_{L}-arabinofuranoside; 6,7-dichloro-3-indolyl-α-_{L}-arabinofuranoside; and 4,6,7-trichloro-3-indolyl-α-_{L}-arabinofuranoside.

13. A cell comprising:
(a) an isolated nucleic acid encoding an α-_{L}-arabinofuranosidase, the nucleic acid being other than a nucleic acid from Streptomyces livians; and
(b) a chromogenic α-_{L}-arabinofuranosidase substrate.

14. The cell of claim 13, wherein the isolated α-_{L}-arabinofuranosidase nucleic acid is from Bacillus subtilis or Aspergillus niger.

15. The cell of claim 13, wherein the chromogenic α-_{L}-arabinofuranosidase substrate comprises an indolyl substituted with a halogen and an α-_{L}-arabinofuranoside.

16. The cell of claim 13, wherein the chromogenic α-_{L}-arabinofuranosidase substrate is a compound having the formula: 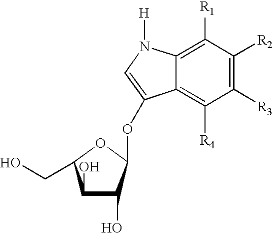
wherein:
R₁, R₂, R₃, and R₄ are independently selected from the group consisting of bromo, chloro, and hydrogen.

17. The cell of claim 13, wherein the chromogenic α-_{L}-arabinofuranosidase substrate is a compound selected from the group consisting of 5-bromo-3-indolyl-α-_{L}-arabinofuranoside; 5-bromo-4-chloro-3-indolyl-α-_{L}-arabinofuranoside; 6-chloro-3-indolyl-α-_{L}-arabinofuranoside; 4,6-dichloro-3-indolyl-α-_{L}-arabinofuranoside; 6,7-dichloro-3-indolyl-α-_{L}-arabinofuranoside; and 4,6,7-trichloro-3-indolyl-α-_{L}-arabinofuranoside.

18. The cell of claim 13, wherein the cell is prokaryotic or eukaryotic.

19. The cell of claim 13, wherein the cell is selected from the group consisting of a bacterial cell, a yeast cell, and a mammalian cell.

20. The cell of claim 13, wherein Cre recombinase is expressed in the cell.

21. The cell of claim 20, wherein the α-_{L}-arabinofuranosidase is expressed in the cell only if the Cre recombinase is expressed.

22. The cell of claim 13, wherein β-galactosidase is expressed in the cell.

23. A cell comprising:
(a) an isolated nucleic acid encoding an α-_{L}-arabinofuranosidase; and
(b) a chromogenic α-_{L}-arabinofuranosidase substrate having formula (I): 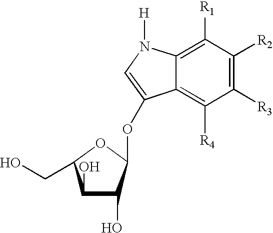
wherein:
R₁, R₂ and R₄ are independently selected from the group consisting of bromo, chloro, and hydrogen; and
R₃ is chloro or hydrogen.

24. The cell of claim 23, wherein the isolated α-_{L}-arabinofuranosidase nucleic acid is from Bacillus subtilis or Aspergillus niger.

25. The cell of claim 23, wherein the chromogenic α-_{L}-arabinofuranosidase substrate is a compound selected from the group consisting of 5-bromo-4-chloro-3-indolyl-α-_{L}-arabinofuranoside; 6-chloro-3-indolyl-α-_{L}-arabinofuranoside; 4,6-dichloro-3-indolyl-α-_{L}-arabinofuranoside; 6,7-dichloro-3-indolyl-α-_{L}-arabinofuranoside; and 4,6,7-trichloro-3-indolyl-α-_{L}-arabinofuranoside.

26. The cell of claim 23, wherein the cell is prokaryotic or eukaryotic.

27. The cell of claim 23, wherein the cell is selected from the group consisting of a bacterial cell, a yeast cell, and a mammalian cell.

28. The cell of claim 23, wherein Cre recombinase is expressed in the cell.

29. The cell of claim 28, wherein the α-_{L}-arabinofuranosidase is expressed in the cell only if the Cre recombinase is expressed.

30. The cell of claim 23, wherein β-galactosidase is expressed in the cell.

31. A cell comprising:
(a) an isolated nucleic acid encoding an α-_{L}-arabinofuranosidase from Bacillus subtilis or Aspergillus niger; and
(b) a chromogenic α-_{L}-arabinofuranosidase substrate.

32. The cell of claim 31, wherein the chromogenic α-_{L}-arabinofuranosidase substrate comprises an indolyl substituted with a halogen and an α-_{L}-arabinofuranoside.

33. The cell of claim 31, wherein the chromogenic α-_{L}-arabinofuranosidase substrate is a compound having the formula: 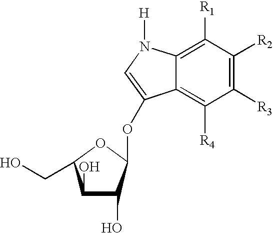
wherein:
R₁, R₂, R₃, and R₄ are independently selected from the group consisting of bromo, chloro, and hydrogen.

34. The cell of claim 31, wherein the chromogenic α-_{L}-arabinofuranosidase substrate is a compound selected from the group consisting of 5-bromo-3-indolyl-α-_{L}-arabinofuranoside; 5-bromo-4-chloro-3-indolyl-α-_{L}-arabinofuranoside; 6-chloro-3-indolyl-α-_{L}-arabinofuranoside; 4,6-dichloro-3-indolyl-α-_{L}-arabinofuranoside; 6,7-dichloro-3-indolyl-α-_{L}-arabinofuranoside; and 4,6,7-trichloro-3-indolyl-α-_{L}-arabinofuranoside.

35. The cell of claim 31, wherein the cell is prokaryotic or eukaryotic.

36. The cell of claim 31, wherein the cell is selected from the group consisting of a bacterial cell, a yeast cell, and a mammalian cell.

37. The cell of claim 31, wherein Cre recombinase is expressed in the cell.

38. The cell of claim 37, wherein the α-_{L}-arabinofuranosidase is expressed in the cell only if the Cre recombinase is expressed.

39. The cell of claim 31, wherein β-galactosidase is expressed in the cell.

40. A transgenic non human organism, the organism having a cell comprising:
(a) an isolated nucleic acid encoding an α-_{L}-arabinofuranosidase, the nucleic acid being other than a nucleic acid from Streptomyces livians; and
(b) a chromogenic α-_{L}-arabinofuranosidase substrate.

41. The transgenic non human organism of claim 40, wherein the isolated α-_{L}-arabinofuranosidase nucleic acid is from Bacillus subtilis or Aspergillus niger.

42. The transgenic non human organism of claim 40, wherein the chromogenic α-_{L}-arabinofuranosidase substrate comprises an indolyl substituted with a halogen and an α-_{L}-arabinofuranoside.

43. The transgenic non human organism of claim 40, wherein the chromogenic α-_{L}-arabinofuranosidase substrate is a compound having the formula: 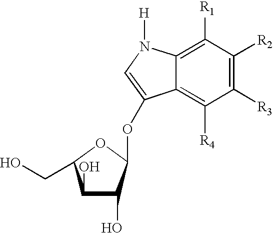
wherein:
R₁, R₂, R₃, and R₄ are independently selected from the group consisting of bromo, chloro, and hydrogen.

44. The transgenic non human organism of claim 40, wherein the chromogenic α-_{L}-arabinofuranosidase substrate is a compound selected from the group consisting of 5-bromo-3-indolyl-α-_{L}-arabinofuranoside; 5-bromo-4-chloro-3-indolyl-α-_{L}-arabinofuranoside; 6-chloro-3-indolyl-α-_{L}-arabinofuranoside; 4,6-dichloro-3-indolyl-α-_{L}-arabinofuranoside; 6,7-dichloro-3-indolyl-α-_{L}-arabinofuranoside; and 4,6,7-trichloro-3-indolyl-α-_{L}-arabinofuranoside.

45. The transgenic non human organism of claim 40, wherein the organism is prokaryotic or eukaryotic.

46. The transgenic non human organism of claim 40, wherein the organism is selected from the group consisting of bacteria, yeast, and mammalian.

47. The transgenic non human organism of claim 40, wherein the organism is a mouse.

48. The transgenic non human organism of claim 40, wherein Cre recombinase is expressed in the cell.

49. The transgenic non human organism of claim 48, wherein the α-_{L}-arabinofuranosidase is expressed in the cell only if the Cre recombinase is expressed.

50. The transgenic non human organism of claim 40, wherein β-galactosidase is expressed in the cell.

51. A transgenic non human organism, the organism having a cell comprising:
(a) an isolated nucleic acid encoding an α-_{L}-arabinofuranosidase; and
(b) a chromogenic α-_{L}-arabinofuranosidase substrate having formula (I): 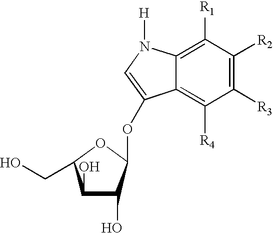
wherein:
R₁, R₂ and R₄ are independently selected from the group consisting of bromo, chloro, and hydrogen; and
R₃ is chloro or hydrogen.

52. The transgenic non human organism of claim 51, wherein the isolated α-_{L}-arabinofuranosidase nucleic acid is from Bacillus subtilis or Aspergillus niger.

53. The transgenic non human organism of claim 51, wherein the chromogenic α-_{L}-arabinofuranosidase substrate is a compound selected from the group consisting of 5-bromo-4-chloro-3-indolyl-α-_{L}-arabinofuranoside; 6-chloro-3-indolyl-α-_{L}-arabinofuranoside; 4,6-dichloro-3-indolyl-α-_{L}-arabinofuranoside; 6,7-dichloro-3-indolyl-α-_{L}-arabinofuranoside; and 4,6,7-trichloro-3-indolyl-α-_{L}-arabinofuranoside.

54. The transgenic non human organism of claim 51, wherein the organism is prokaryotic or eukaryotic.

55. The transgenic non human organism of claim 51, wherein the organism is selected from the group consisting of bacteria, yeast, and mammalian.

56. The transgenic non human organism of claim 51, wherein the organism is a mouse.

57. The transgenic non human organism of claim 51, wherein Cre recombinase is expressed in the cell.

58. The transgenic non human organism of claim 57, wherein the α-_{L}-arabinofuranosidase is expressed in the cell only if the Cre recombinase is expressed.

59. The transgenic non human organism of claim 51, wherein β-galactosidase is expressed in the cell.

60. A transgenic non human organism, the organism having a cell comprising:
(a) an isolated nucleic acid encoding an α-_{L}-arabinofuranosidase from Bacillus subtilis or Aspergillus niger; and
(b) a chromogenic α-_{L}-arabinofuranosidase substrate.

61. The transgenic non human organism of claim 60, wherein the chromogenic α-_{L}-arabinofuranosidase substrate comprises an indolyl substituted with a halogen and an α-_{L}-arabinofuranoside.

62. The transgenic non human organism of claim 60, wherein the chromogenic α-_{L}-arabinofuranosidase substrate is a compound having the formula: 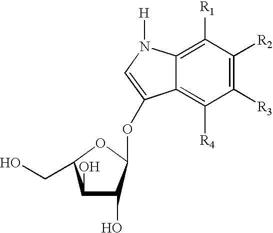
wherein:
R₁, R₂, R₃, and R₄ are independently selected from the group consisting of bromo, chloro, and hydrogen.

63. The transgenic non human organism of claim 60, wherein the chromogenic α-_{L}-arabinofuranosidase substrate is a compound selected from the group consisting of 5-bromo-3-indolyl-α-_{L}-arabinofuranoside; 5-bromo-4-chloro-3-indolyl-α-_{L}-arabinofuranoside; 6-chloro-3-indolyl-α-_{L}-arabinofuranoside; 4,6-dichloro-3-indolyl-α-_{L}-arabinofuranoside; 6,7-dichloro-3-indolyl-α-_{L}-arabinofuranoside; and 4,6,7-trichloro-3-indolyl-α-_{L}-arabinofuranoside.

64. The transgenic non human organism of claim 60, wherein the organism is prokaryotic or eukaryotic.

65. The transgenic non human organism of claim 60, wherein the organism is selected from the group consisting of bacteria, yeast, and mammalian.

66. The transgenic non human organism of claim 60, wherein the organism is a mouse.

67. The transgenic non human organism of claim 60, wherein Cre recombinase is expressed in the cell.

68. The transgenic non human organism of claim 67, wherein the α-_{L}-arabinofuranosidase is expressed in the cell only if the Cre recombinase is expressed.

69. The transgenic non human organism of claim 60, wherein β-galactosidase is expressed in the cell.

70. A vector comprising an insertion site for a regulatory element operably linked to an isolated nucleic acid encoding α-_{L}-arabinofuranosidase, the nucleic acid being other than a nucleic acid from Streptomyces livians.

71. A vector comprising an insertion site for a regulatory element operably linked to an isolated nucleic acid encoding α-_{L}-arabinofuranosidase from Bacillus subtilis or Aspergillus niger.

72. A method to monitor the transcriptional activity of a regulatory sequence in a cell, the method comprising:
(a) introducing into the cell a vector comprising the regulatory sequence operably linked to a nucleic acid encoding α-_{L}-arabinofuranosidase, wherein the nucleic acid is other than a nucleic acid from Streptomyces livians; and
(b) detecting the activity of α-_{L}-arabinofuranosidase in the cell, wherein the activity of α-_{L}-arabinofuranosidase is directly proportional to the transcriptional activity of the regulatory element.

73. The method of claim 72, wherein the α-_{L}-arabinofuranosidase nucleic acid is from Bacillus subtilis or Aspergillus niger.

74. The method of claim 72, wherein the cell is prokaryotic or eukaryotic.

75. The method of claim 72, wherein the cell is selected from the group consisting of a bacterial cell, a yeast cell, and a mammalian cell.

76. The method of claim 72, wherein Cre recombinase is expressed in the cell.

77. The method of claim 76, wherein the α-_{L}-arabinofuranosidase is expressed in the cell only if the Cre recombinase is expressed.

78. The method of claim 72, wherein β-galactosidase is expressed in the cell.

79. The method of claim 72, the method further comprising detecting the activity of α-_{L}-arabinofuranosidase by contacting the cell with a sufficient amount of a chromogenic α-_{L}-arabinofuranosidase substrate, the substrate, when contacted with α-_{L}-arabinofuranosidase forms a colored product, wherein the level of α-_{L}-arabinofuranosidase activity is directly proportional to the amount of colored product formed.

80. The method of claim 79, wherein the chromogenic α-_{L}-arabinofuranosidase substrate comprises an indolyl substituted with a halogen and an α-_{L}-arabinofuranoside.

81. The method of claim 79, wherein the chromogenic α-_{L}-arabinofuranosidase substrate is a compound selected from the group consisting of 5-bromo-3-indolyl-α-_{L}-arabinofuranoside; 5-bromo-4-chloro-3-indolyl-α-_{L}-arabinofuranoside; 6-chloro-3-indolyl-α-_{L}-arabinofuranoside; 4,6-dichloro-3-indolyl-α-_{L}-arabinofuranoside; 6,7-dichloro-3-indolyl-α-_{L}-arabinofuranoside; and 4,6,7-trichloro-3-indolyl-α-_{L}-arabinofuranoside.

82. The method of claim 72, wherein the vector is introduced into the cell by liposomal mediated transfection.

83. A method to monitor the transcriptional activity of a regulatory sequence in a cell, the method comprising:
(a) introducing into the cell a vector comprising the regulatory sequence operably linked to a nucleic acid-encoding α-_{L}-arabinofuranosidase from Bacillus subtilis or Aspergillus niger; and
(b) detecting the activity of α-_{L}-arabinofuranosidase in the cell, wherein the activity of α-_{L}-arabinofuranosidase is directly proportional to the transcriptional activity of the regulatory element.

84. The method of claim 83, wherein the cell is prokaryotic or eukaryotic.

85. The method of claim 83, wherein the cell is selected from the group consisting of a bacterial cell, a yeast cell, and a mammalian cell.

86. The method of claim 83, wherein Cre recombinase is expressed in the cell.

87. The method of claim 86, wherein the α-_{L}-arabinofuranosidase is expressed in the cell only if the Cre recombinase is expressed.

88. The method of claim 83, wherein β-galactosidase is expressed in the cell.

89. The method of claim 83, the method further comprising detecting the activity of α-_{L}-arabinofuranosidase by contacting the cell with a sufficient amount of a chromogenic α-_{L}-arabinofuranosidase substrate, the substrate, when contacted with α-_{L}-arabinofuranosidase forms a colored product, wherein the level of α-_{L}-arabinofuranosidase activity is directly proportional to the amount of colored product formed.

90. The method of claim 89, wherein the chromogenic α-_{L}-arabinofuranosidase substrate comprises an indolyl substituted with a halogen and an α-_{L}-arabinofuranoside.

91. The method of claim 89, wherein the chromogenic α-_{L}-arabinofuranosidase substrate is a compound selected from the group consisting of 5-bromo-3-indolyl-α-_{L}-arabinofuranoside; 5-bromo-4-chloro-3-indolyl-α-_{L}-arabinofuranoside; 6-chloro-3-indolyl-α-_{L}-arabinofuranoside; 4,6-dichloro-3-indolyl-α-_{L}-arabinofuranoside; 6,7-dichloro-3-indolyl-α-_{L}-arabinofuranoside; and 4,6,7-trichloro-3-indolyl-α-_{L}-arabinofuranoside.

92. The method of claim 83, wherein the vector is introduced into the cell by liposomal mediated transfection.

93. A method to monitor the transcriptional activity of a first regulatory sequence and a second regulatory sequence in a cell, the method comprising:
(a) introducing into the cell a first vector comprising the first regulatory sequence operably linked to a nucleic acid encoding α-_{L}-arabinofuranosidase from Bacillus subtilis or Aspergillus niger;
(b) introducing into the cell a second vector comprising the second regulatory sequence operably linked to a nucleic acid encoding β-galactosidase;
(c) detecting the activity of α-_{L}-arabinofuranosidase in the cell, wherein the activity of α-_{L}-arabinofuranosidase is directly proportional to the transcriptional activity of the first regulatory element; and
(d) detecting the activity of β-galactosidase in the cell, wherein the activity of β-galactosidase is directly proportional to the transcriptional activity of the second regulatory element.

94. The method of claim 93, wherein the cell is prokaryotic or eukaryotic.

95. The method of claim 93, wherein the cell is selected from the group consisting of a bacterial cell, a yeast cell, and a mammalian cell.

96. The method of claim 93, wherein Cre recombinase is expressed in the cell.

97. The method of claim 96, wherein the α-_{L}-arabinofuranosidase is expressed in the cell only if the Cre recombinase is expressed.

98. The method of claim 93, the method further comprising:
(a) detecting the activity of α-_{L}-arabinofuranosidase by contacting the cell with a sufficient amount of a chromogenic α-_{L}-arabinofuranosidase substrate, the substrate, when contacted with α-_{L}-arabinofuranosidase forms a colored product, wherein the level of α-_{L}-arabinofuranosidase activity is directly proportional to the amount of colored product formed; and
(b) detecting the activity of β-galactosidase by contacting the cell with a sufficient amount of a chromogenic β-galactosidase substrate, the substrate, when contacted with β-galactosidase forms a colored product, wherein the level of β-galactosidase activity is directly proportional to the amount of colored product formed.

99. The method of claim 98, wherein the chromogenic α-_{L}-arabinofuranosidase substrate forms a blue colored product when contacted with α-_{L}-arabinofuranosidase and the chromogenic β-galactosidase substrate forms a red colored product when contacted with β-galactosidase.

100. The method of claim 98, wherein the chromogenic α-_{L}-arabinofuranosidase substrate forms a red colored product when contacted with α-_{L}-arabinofuranosidase and the chromogenic β-galactosidase substrate forms a blue colored product when contacted with β-galactosidase.

101. The method of claim 98, wherein the chromogenic α-_{L}-arabinofuranosidase substrate comprises an indolyl substituted with a halogen and an α-_{L}-arabinofuranoside.

102. The method of claim 98, wherein the chromogenic α-_{L}-arabinofuranosidase substrate is a compound selected from the group consisting of 5-bromo-3-indolyl-α-_{L}-arabinofuranoside; 5-bromo-4-chloro-3-indolyl-α-_{L}-arabinofuranoside; 6-chloro-3-indolyl-α-_{L}-arabinofuranoside; 4,6-dichloro-3-indolyl-α-_{L}-arabinofuranoside; 6,7-dichloro-3-indolyl-α-_{L}-arabinofuranoside; and 4,6,7-trichloro-3-indolyl-α-_{L}-arabinofuranoside.

103. The method of claim 93, wherein the first and second vector are introduced into the cell by liposomal mediated transfection.

104. A chromogenic α-_{L}-arabinofuranosidase substrate having an indolyl substituted with a halogen and an α-_{L}-arabinofuranoside, the compound when contacted with a α-_{L}-arabinofuranosidase forms a colored product, wherein the compound is other than 5-bromo-3-indolyl-α-_{L}-arabinofuranoside.

105. A chromogenic α-_{L}-arabinofuranosidase substrate having formula (I): 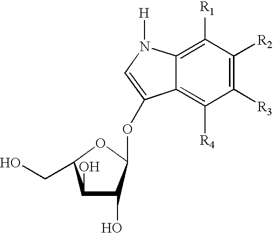
wherein:
R₁, R₂ and R₄ are independently selected from the group consisting of bromo, chloro, and hydrogen; and
R₃ is chloro or hydrogen.

106. The chromogenic α-_{L}-arabinofuranosidase substrate of claim 105, wherein the chromogenic α-_{L}-arabinofuranosidase substrate is a compound selected from the group consisting of 5-bromo-4-chloro-3 -indolyl-α-_{L}-arabinofuranoside; 6-chloro-3 -indolyl-α-_{L}-arabinofuranoside; 4,6-dichloro-3-indolyl-α-_{L}-arabinofuranoside; 6,7-dichloro-3-indolyl-α-_{L}-arabinofuranoside; and 4,6,7-trichloro-3-indolyl-α-_{L}-arabinofuranoside.

107. A kit for monitoring the transcriptional activity of a regulatory sequence, the kit comprising:
(a) a vector comprising an insertion site for the regulatory element operably linked to a nucleic acid encoding α-_{L}-arabinofuranosidase;
(b) a chromogenic α-_{L}-arabinofuranosidase substrate;
(c) instructions for detecting the activity of α-_{L}-arabinofuranosidase.

108. A composition for use in monitoring transcriptional activity of a regulatory sequence, the composition comprising:
(a) a chromogenic α-_{L}-arabinofuranosidase substrate having an indolyl substituted with a halogen and an α-_{L}-arabinofuranoside, wherein the compound is other than 5-bromo-3-indolyl-α-_{L}-arabinofuranoside; and
(b) a chromogenic β-galactosidase substrate.

109. The composition of claim 107, wherein the chromogenic α-_{L}-arabinofuranosidase substrate is a compound having formula (I): 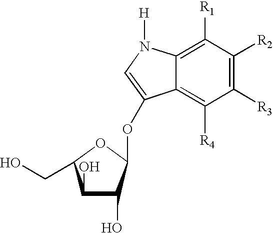
wherein:
R₁, R₂ and R₄ are independently selected from the group consisting of bromo, chloro, and hydrogen; and
R₃ is chloro or hydrogen.

110. The composition of claim 107, wherein
(a) the chromogenic α-_{L}-arabinofuranosidase substrate is a compound selected from the group consisting of 5-bromo-4-chloro-3-indolyl-α-_{L}-arabinofuranoside; 6-chloro-3-indolyl-α-_{L}-arabinofuranoside; 4,6-dichloro-3-indolyl-α-_{L}-arabinofuranoside; 6,7-dichloro-3-indolyl-α-_{L}-arabinofuranoside; and 4,6,7-trichloro-3-indolyl-α-_{L}-arabinofuranoside; and
(b) the chromogenic β-galactosidase substrate is a compound selected from the group consisting of 5-bromo-4-chloro-3-indolyl-β-galactosidase, 6-chloroindolyl-β-galactosidase, 4,6-dichloroindolyl-β-galactosidase,6,7-dichloroindolyl-β-galactosidase, and 4,6,7-trichloroindolyl-β-galactosidase.


SEQUENCE LISTING (3 sequences)

SEQ ID NO: 1 - DNA, 2530 bp, Streptomyces lividans
ggccgggcgcctgatggccggcctgccgctgatcgtcgtcttcgtcttcttccagcgccagatcgtggcggcgtcgcccacacggcctggccggccagtagcccctcaccccgtcccgaaaggaaaagatgcgcaccgcccgcttcaccctcgaccccgccttcaccgtcggcgccgtcaacccccgtctcttcggctccttcgtcgaacacctcggacgctgcgtgtacaccggcgtcttcgagcccggccaccccaccgcggacgccgagggcctgcgccaggacgtgctggagctggtccgggaactcggcgtcaccgccgtccgctaccccggcggcaacttcgtctccggctacaagtgggaggactcggtcggcccggtggaggaccgtccgcgccgcctcgacctggcctggcgctccaccgagaccaaccgcttcggcctctccgagtacatcgccttcctgaagaagatcggcccgcaggccgagcccatgatggcggtcaacctcggcacccgcggcgtcgccgaggcgctcgaactccaggagtacgccaaccacccctccggcacggccctgtccgacctgcgcgccgagcacggcgacaaggaccccttcggcatcaggctctggtgcctgggcaacgagatggacggcccctggcagaccggccacaagaccgccgaggagtacggccgcgtcgccgccgagaccgcccgcgccatgcgccagatcgacccggacgtcgaactggtcgcgtgcgggtcgtccgggcagtccatggagaccttcgccgagtgggaggcgacggtcctgaaggagacctacgacctggtcgaccacatctcgctgcacgcctactacgagccgcacgacggggacgtcgactccttcctcgcctccgccgtggacatggagtcgttcatcgagaacgtcgtcgccacctgcgaccacgtcggcgcccgcctcaagtcgaagaagaagatcaacctctccttcgacgagtggaacgtctggtacatgaccaagacgcaggccgaggtcagcgccctggactggcccgaggcgccccgcctgctcgaggacaactacagcgtcatggacgcggtcgtcttcggctcgctcctgatcgccctgctgcggcacgccgaccgcgtcaccgtggcctgcctcgcgcagctcgtcaacgtcatcgcgccgatcatgaccgagccgggcggcccggcctggcgccagacgacgttcttccccttctcccaggcctcgaagtacggccgcggcgaggtcctcgacgtccgcgtggactccccgacgtacgacaccgcgaagtacggcgaggccgacctgctgcacgccaccgccgtggtgcgcgccagacgctcggtcaccgtcttcgccgtcaaccgctcgcgcaccggggccctcccgctggaggtcgccctgagcggcctggagctgaccgaggtcgtcgagcacagcgccctcgcggacgccgaccccgacgcccgcaacaccctcgccgagcccgagcgcgtcgtcccgcacccggtcgacggcacctccctgagggacggccggctcaccgccgccctggaaccgctgtcctggaactcgatccgctgcgctgacccggccccgggtcagccgccgcgccggccgggggaggggacgggcttcaccggcactcctccggccgcgccgcccagcagctccagcgcaccgcgcccggacccgacggcgcgccgttcgccggacaggaccgcgagggccagggtgttggcggccgcccgggtgcgcaggatgccgttcggcaggacgaaggtgtgcggggcacccgtgcggccgcccacgtacgcgcccaggttccagccgttcaggaagacctggacgcggtgggcaccggcgccgcggtccgggtctccgtccaggcgcagccccaccgaggcgtcgatccccggcgggacgtccagccggaacgtggtccggtaccaggtcacgccctggcgccggtccccacccggcagcgcgccggggacacccgctcccacccgccgtcgtcgtacccgggcaggtgccagccgtggcgctccccgtacagcccgccgttgttgagcggcccgcgcaccgggtcgggggcggccgcgcccatgatccgccagcgcacctcgggcgaggcgccctcgaacgccgccgacgtcagcccgcgggcctcccggtacgcgtcctggccgtgctgtgtgcgccgcaccaggacggacaggacggggccgtcgtccccttcgccgcccttgctccccccgcccgccctgctcccggcggcccgacgcagtgcggcgcgcaggtcctccggcagcccgaacgcggcgtgccggtccacgtgcccttcccgtcgcccgccccgtccggctggtggctgcccagcggcaccccgtccagccaggccatcagcaggccgtccgcgcccgtgctgtacgcgagggacacccgctccagccccgtggcgccggtgaggcgggcgcggtacc

SEQ ID NO: 2 - DNA, 6811 bp, Bacillus subtilis
atggccagtcatgatacgcctgtgtcaccggctggcattctgattgacttggacggtactgtattcagaggaaatgagttgatcgaaggagcaagagaagcgatcaaaacgcttaggagaatgggaaagaaaatcgtctttttaagcaaccgggggaatatctcccgtgccatgtgcagaaaaaaacttcttggcgcggggattgaaacggacgtaaacgacattgttctgtcatcaagcgtgacagcggcttttctgaaaaaacattatcgtttttcaaaggtatgggtgcttggggagcaaggcttggttgacgagctgaggctggccggtgtgcagaacgcgagcgaaccgaaggaagcggattggctcgtgatctcccttcatgaaacgctcacgtacgacgatttaaatcaagcctttcaagcggctgccggcggcgctcgtattatcgctacaaacaaagaccgctcttttccgaacgaagacggaaatgccattgatgtggccggaatgatcggggcaattgagacttctgcacaagcgaagactgaacttgttgtcggaaaaccgtcatggctgatggcggaggctgcctgtacggcaatggggctgtccgcacatgaatgcatgattataggagacagcattgaatctgacattgcgatggggaagctttatggcatgaaaagcgccttagtgctaactggttctgcgaaacagggtgaacagcgtttgtacacgccggattatgtgctggattctattaaggatgtaaccaaattggctgaggaggggattctgatatgaatcgtatcgcagctgacgttcagcgtgcttttgaaaacgccggagaaaagacgttgcctataaaagttgaagaaattgttctcggtaagcaagcagctgattcgcttttggattatgtaaaacgaaaaaacaatcaacatattgtccttgtctgcgacgcgaatacacaccgcattgcaggaattgatttagaaaaccgactgaatcaagaaggatttcaggccgagtgcctgatcattccagaaaatgaagccggagatgtgacagctgatgaacgatcgctcattcatgtgctgatccatacgaaacaaccaacggatgtcatgatcgcagtcggttcgggcacgattcatgatatcgtccgctttgcggcgtttcaaagagatttgccgtttatttcttatccgactgctccatctgtagacggttttacatcagccggtgcgccgattattttatacggcacgaaaacaaccattcaaacgaaggccccatctgcgctgttcgctgatctggatctattaaaagcggcaccgcagtcaatggtggcggctggctttggtgacatgctcggtaaaatcacgtctttagcagattgggaaatatcccggcatcttgccggtgagccttattcgcctgcaggagctaagatcgttcaggaggcgcttgctgcctgcattgaacacacagaagacattgcgatgaaaacggaaactggcatacgggttttgatggagtctttacttgtatcggggcttgtcatgctggctttagatcattcccgaccggcatcaggcggcgagcatcatatttcacattggattgaaatggagttaatggagaaaaaacggcctcagattcttcatggggcaaaggtgggctgtgccgctgttttattaactgacacatacagaaagctcgctcaggatgacgggctgaacgaattttcaccaagccgccgggaagccatccaatcggcttatcaaacactcccgagaggagaagtgctggctgattggctgagatcagccggaggccctgctgattttgacgaaatcggtgtcgggcaggattccgtcaaaaatgccttcagacacgcgcacaccttaagagaccgatgcaccggattaagaatcatcaatgaaaacaaaacgctgatcaaccatggtctatatgaatagcccgcacctcgaatggaaggggtaacgcagatgaaaaaaatgactgtctgttttcttgtgctcatgatgttgctgacattagtcattgccgggtgttcagcagaaaaatcatccggcaaatcgggtgaaactgagctgaccttttggacatttaacgggcttcatgagcagttctatgtggaaatggtgaaggaatggaacaaaaaatatcctgaccgcaaaattaagctgaatacggtcgtttatccatatggacaaatgcacgataacttatctatctccctaatagcgggagaaggcgttcctgatattgcagatgtcgaattggcccgtttttcaaactttttgaagggctctgacataccgcttgccgacttgactccgctgattgaaaaggatcgcgataaattcgttgaggcgcggctgacattgtacagcaaaaacggaaagctttacggactcgatacacatgtagggacaacggtcatgttttataacatggatgtgatgaaaaaagccggcgtcaatcctgacgatattaaaacatgggatgattaccataaagccggacagaaagtgcgcaaagtgaccgggaagccgatgggaacggtggaaacaaatgattccgcaacgttcttatctatgatttcacagcaaaactcaggctattttgataaaaacggcaagctgatcctcaataatgacaccaacgtaaaaacacttcaatatttaaaagacatgatcaatgataaaacgatgattcctgcgccgggcggcgggcatcacagtgaagaatactacggctttatgaaccaaggaggagctgcttcagttctcatgccgatttggtatatgggaagatttatcgattatatgcctgatctgaaagggaagattgccatcagaccgctcccggcatggaaagaggggggcgaccgctcggcaggtttgggcggtacggcaactgttgtaccgaagcaatccaagcatgttgagttagcaaaagagtttttggcctttgcgaagggctctgaagaaggaaataaaaaactctggagcgtactcggctttgacccgcttcgctgggatgtttggagctccaaggaattgaaagagaaaaacaaatacacggattacttccaaaacggaacaggcattttttctgtgctgctcgatatcaaggatgaaatcaatccaatttatttacatgaggattttgccaaggcttcagaccttgtcaacagaagcgtattgttcgacgcgcttaaatctcagcaaaaaacgcctaaacaagccttggacagagcggcaggtgaactgaaacagaaatagaatcccattcaaaaagtgaaagcggggaggttctcatgaaacctgtgaaaacgggaacggttcatcccgttccttcagctgcgaaacaatcaggctggcgagatctgttttattcaaaaaaagcggcgccctatctgtttacagcgccattcgttttatcctttctcgtattttttctataccccatcattagtgtcttcatcatgagcttccaaagaattttgccgggagaggtgtcctttgtcggattgtctaattatacagcgctaaacaacccgacgttctataccgccctttggaatacgctggaatacaccttttggacgctgatcgtgctgattcctgttccattgcttctggccatattcctgaattcaaagctggtcaaatttagaaatatatttaaatcagcattatttatcccggcattgacctcaaccattgtcgcggggatcatttttcggctgatcttcggagaaatggaaacgtctctggccaattccatcctacttaaactcggcttttcacctcagaactggatgaacaatgaacataccggcatgtttttgatggtgctgcttgcttcatggaaatggatgggaatcaacatcctttactttttagcaggtttgcaaaatgtgccgaaagagctgtacgaagccgctgatatagacggcgcgaatacaatgaaaaaatttctgcacatcacgctgccgtttctcaagcctgtaaccgtatatgtgctgaccatcagcatcatcggcggcttcaggatgtttgaggaaagctacgtcctttggcagaataattccccgggtaatattggtctgacgcttgtcggatatttgtatcagcagggacttgcctacaatgaaatgggatacggagcggccatcggcattgtgcttttgattgtgatccttgttgtcagcctgatttcattaaagctgtcaggctcgtttaagggggagggataaatgttgcggcacagtcctcagtttagcgtttatagaattgcgctgaccctgttttttatgatgctgagcctattgtatctttttccgattttctgtttgcttttaggatcattaaagccgtcatctgagcttttgcgtgtggggctgaatcttgatattgatccaaaagtgatgagttttgataactacacatttctgtttaatggcggcagcatttatttcaaatggttttttaacagtcttgtactcggactttttacgactgtgctcactctgtttttttcttcgatgatcgggtacgggcttgcggtttatgattttaagggcagaaatatcatctttgttcttgtgctgattattatgatggttccgctggaagtgatgatgcttcctctgtttaaacttactgtcggactgcacttgatcgattcatatacgggtgtcatattgccgtttatcgtttcacctgttgctgttttctttttcaggcaatatgctcttggccttccaagagatctgctggactctgcaaggatggacggctgtacggaattcggcatctttttcaggattatggcaccgctgatgaaaccggctttcggtgcgatgattatccttcagtccttaaacagctggaacaacttcttgtggccgctgattgtgcttcggtcgaaagaaatgtttacgcttccaatagggctgtccagcttgctgagcccttatggaaataattacgacatgcttatatccggctcagtatttgcgattttgccggtgattatcattttcttgtttttccaaaagtactttatctccggcctgacggtagggggagtcaaaggttaatgaaggaggaaacgtgtgatgaaaaaagcgcgaatgattgtagacaaagaatataaaatcggtgaagtagataaacggatttatggctcgtttatcgaacatatgggtcgtgcggtatatgaaggcatatacgagcctgatcaccctgaagcggatgaagatggatttagaaaagatgtccagtcgctgatcaaagaattacaggttcccatcatccgctatccgggcggaaactttttatccggatacaactgggaggacggtgtcggaccagtcgaaaaccgcccgagacggcttgacttggcatggcaaacgacagaaaccaatgaagtgggaacaaatgaatttttatcttgggcaaaaaaggtgaacactgaggtcaatatggccgtcaaccttggcacaagaggcatagatgccgcccgtaatctcgttgaatattgcaaccatccgaaaggctcttactggagtgatttaagaagatcgcatggctatgaacagccgtatggcatcaaaacatggtgcttaggaaacgaaatggatggaccatggcagatcggccacaaaacagctgatgaatacggacggcttgccgcagagacagcaaaggtcatgaagtgggttgacccatcaattgaactcgttgcctgcggcagctcaaacagcggtatgccgacctttatcgattgggaagcgaaggtgcttgagcatacgtatgagcatgtcgactatatctctcttcacacttactacggaaaccgggataacaatctgccaaactacttggcacgttctatggatttggatcattttatcaaatcagtcgctgcgacctgtgactatgtaaaagcaaaaacacgcagcaagaaaactatcaatctctctctggatgaatggaacgtctggtaccactcaaatgaggctgataaaaaagtcgagccgtggatcactgcgcgtccgattttagaggatatttacaattttgaagatgccttattagtcggctctctgctcattacgatgctgcagcacgcagaccgtgtgaaaattgcgtgtcttgcacagcttgttaatgtcatcgcgccgatcatgacggaaaaaggcggagaagcatggagacagccgattttctatccatacatgcatgcttctgtttacggaaggggcgagtcactgaaaccgcttatttcttctcctaagtacgattgttctgatttcactgatgtgccatatgttgatgctgctgttgtgtactctgaagaggaagaaacactcactatttttgcggtaaacaaggctgaggatcagatggagacggagatttcgctcagaggctttgaatcctaccaaatcgcagagcacatcgtacttgagcatcaggatatcaaagcaacaaaccagcataacagaaaaaatgtcgttccgcattccaacggatcatcgtctgtcagcgaaaacggcttaactgctcatttcacgccgctttcctggaatgtgatccgcctgaaaaaacagtcataagaatagcaaagccggagatttctctccggcttgtctttcaactgccacgagccggcccattccagccggctttttgtataggaaaaaatgaccgcttttcaccatgaaattatgatatatttatgaaaaacagaaaaggggatg

SEQ ID NO: 3 - DNA, 4122 bp, Aspergillus niger
ggatcccttccagaaagataagtgggagaagtaccgccgcgagattttacaaaatggtggcagccagcaagacctgttccagatgctggagagatttttgggtcgtccgccaaatatgaatgctcttgtggaaggtctgaagcgggcagacagtcaacacgaatgttagaagaggcggcatattacaccatgggcttttcagacagaattactgcagaagtcagagccacgcaatgttaccatgccacgccaatcaggtcagctgcttgaattgggttcaaaatccccaggaacccgagcggcccgagcctgccgagggtcacagacgatgcggagggccgcttggcatccaatgtctccctctttataactcccatatcatgtctgccttggccatataagccattccatagtgcaatgccatagactgtctatcggaaggtgtccggtaattctccgtgtttcttacagatgctcgggaagtcaatagactgcagggctcttcatgcgtcatgatataaccgacggcacctcctttgggctgtgcttagtgggtccggtaggctagacattcaagcgcagccattcacagcttcagtccaaagacatgaccgaatagtagtatcctaatagagttcgagtaccatgaccacatggtgtgaagaacatgggttcccctcacggcgccggttaaaggccatcccctcctcaaagaataaacatcgtacgacgtacacgggtcggttgtttcaactattatatttcattccaactatacttattgcaaagcggaattggctccggtcatcatacagcctttcaggggtaatcatattgctcgatatccggggtaatgacatccaagttgtacctactaaaacttacccattactcgaaagtagacactgcctccggaaaagcattcaacaaccctcaacaaaactaacttccgcaggaatgcggcacctccattccctccacaattaccgaggcttatcccaggaggaacagatccctgaagaaacgtgacgaaccgtgatgagcgggcaatcacagattagtctcttcccccgcatccccgacgggcaaaaccccggtggcactggcattagccctaggcacggacaaagccattgtcaagccttcaggaactaaatcaactaattctccccatccctgcggggtacatactccacgacgccctgcacgcccaccggaatgccgtcgtcgcttgacatcatgtggaacaacacattccttctgcctcaaaactgagccttttaaccgaccggttacttaacaatgtatacaggaggtctcaatattggaggaaaatccctgacccctgaagccacacatgtaactagacttttgggatcttgcatttcatgcataggcactgactgcagtaaagaatataaataagcctcccttcgcaccctagtagcatgatttctttctccaagcggcctatctcactgtctcctctcctagcccagaacttactgagcaggcagtaatcctccaccatgttctcccgccgaaacctcgtcgccctagggctggcagccaccgtcagcgccggcccctgtgacatctacgaagccggcgacacgccctgcgtagccgcgcacagcaccacccgcgccctatacagctccttcagcggcgccctctaccagctccaacgtggctccgacgacaccaccaccaccatctccccgctcacagccggcggcgtcgccgacgcctccgcccaagacaccttctgtgccaacaccacctgcttgatcaccatcatctacgaccaatccggcaacggcaaccacctgacccaagcacccccgggaggcttcgacggccccgacgtcgacggctacgacaacctcgccagcgccatcggcgcccccgtcaccctcaatggccaaaaggcctacggcgtgttcatgtcccccggcacgggctaccgtaacaacgaagccaccggcaccgccaccggcgacgaacccgaaggcatgtacgccgtcctggacggcacgcactacaacgacgcctgctgcttcgactacggcaacgccgagaccagcagcaccgacactggcgccggccacatggaggccatctacctcgggaacagcaccacctggggctacggcgccggtgacgggccctggatcatggtcgatatggagaacaacctcttctccggtgctgatgagggatataactccggagatccctcgatcagctacagctttgtcactgccgcggtcaagggcggggctgataagtgggctattcgcggtggtaatgctgcctctgggtccctctctacttactacagcggcgctcgcccggattactccggctataaccccatgagcaaggagggcgctatcatcctgggtatcggcggtgacaacagcaacggcgcccagggtaccttctacgagggtgtcatgacctccggctacccgtcggacgatgtcgagaactccgtccaggagaacatcgtggctgcgaaatacgtctccggctcgctggtcagcggcccgtcgttcacctccggagaggtggtctcgctgcgtgtcactacccccggttacacgacgcggtatattgcgcacactgacaccactgtgaacacgcaggtcgtggacgacgatagttccaccacgctgaaggaggaggctagctggaccgtggtgacaggtctggctaatagtcagtgcttctcgttcgagtcggttgatacccctggtagctatatccggcattacaactttgagttgctgcttaatgccaacgatggcacgaagcagttccatgaggatgctactttctgtcctcaggcgccgttgaatggagaaggtacttcgttgagatcgtggagttatccgacgaggtatttcaggcattatgagaatgtcctgtatgctgctagtaatggtggtgtgcagacgtttgattccaagacgtcgtttaataatgatgttagctttgagattgagacggcgtttgcttcgtaaggggggaattgggggttgtttggtggtttgggtgtggggttgtatctgcctgtgttggcggggagagtggtttgtaaataggtcatttttggtatatagcatacgaatactatacgatagtatacatgatagtggttgtttcatgtaatcgagaaattattcaaggcttgcaatacaatatcttattactgtatctcgtggagcattcacagactgaacggctgcacaatgattcttatgcggtgatattgactgtgattagatgatatattcaggtgtgctatttttcttgtatttatgcttcatgcgaatatatgtatttcacgacagacagagaggttaaacaaagcatactaacgagcgattcagatatggcatgtatgaataagacaaaagtactgcgacactcaagaccaacacaaaccagtatacttcataacaatatatgctaggcagtacctccgattctacgatgagctttaccagaaaatggatttctttgtccctcgtcattctcaatttttgggccttccacggctgcggcaacctgttgagcaagacgtgttctctcgtacggatggacgttgatcttcttctcgagcaggtccaattgctctttctccaaatggcggactctgacctcaccggaggaatcactcggcaagcatatctggtaagtcgctgaaaatttcttctcgaaactggcgtagcttggtggccactgcgggaagcgtcggaagtatttgcctgtccagacggtcaatatgaatgagtcggatacttcaatgcccgcctgccttctacggtcgaagaaatgctggccgatgcttctcacggaggcatagtttataacaggcttgatggggttttgtatgtcgaggagacctaattcaaagtgcatagataggctcctgagattgtgaaaaccggttgctagtttgtcgaagacgtcgttgggctgttgtgtggttatagttcagttcatgggatgtactaagaggattctttggcttacccattgatcgttctctctcttgactcccacctgcagtgacttcagtttgggacagctaacatcgaggtggctgagctc